(19) United States
(12) Patent Application Publication
(10) Pub. No.: US 2010/0021985 A1
(43) Pub. Date: Jan. 28, 2010

(54) MECHANICAL PROCESS FOR CREATING PARTICLES IN FLUID

(75) Inventor: Thomas G. Mason, Los Angeles, CA (US)

Correspondence Address: VENABLE LLP, P.O. BOX 34385, WASHINGTON, DC, 20043-9998, US

(73) Assignee: The Regents of the University of California, Oakland, CA (US)

(21) Appl. No.: 12/563,907

(22) Filed: Sep. 21, 2009

Related U.S. Application Data
(63) Continuation-in-part of application No. PCT/US2008/003679, filed on Mar. 20, 2008.
(60) Provisional application No. 61/100,471, filed on Sep. 26, 2008.
(60) Provisional application No. 60/918,896, filed on Mar. 20, 2007.

(51) Int. Cl. B05D 7/00; B05C 11/00; C23C 14/34; C09D 11/00; H01F 1/00; C09K 3/00; C08K 9/00; C09D 189/00; C09D 103/00; C12N 11/00; C12N 7/00

(52) U.S. Cl. 435/174; 118/72; 427/212; 204/192.15; 427/580; 106/31.13; 252/62.51 R; 252/625; 523/200; 106/124.1; 106/162.1; 435/235.1

(57) ABSTRACT

A method of producing at least one of microscopic and submicroscopic particles includes providing a template that has a plurality of discrete surface portions, each discrete surface portion having a surface geometry selected to impart a desired geometrical property to a particle while being produced; depositing a constituent material of the at least one of microscopic and submicroscopic particles being produced onto the plurality of discrete surface portions of the template to form at least portions of the particles; separating the at least one of microscopic and submicroscopic particles comprising the constituent material from the template into a fluid material, the particles being separate from each other at respective discrete surface portions of the template; and processing the template for subsequent use in producing additional at least one of microscopic and submicroscopic particles. A multi-component composition includes a first material component in which particles can be dispersed, and a plurality of particles dispersed in the first material component. The plurality of particles is produced by methods according to embodiments of the current invention.

### CROSS-REFERENCE TO RELATED APPLICATION

This application claims priority to U.S. Provisional Application No. 61/100,471 filed Sep. 26, 2008. This application is a Continuation-in-Part of PCT/US2008/003679, filed Mar. 20, 2008, which claims priority to U.S. Provisional Application No. 60/918,896, filed Mar. 20, 2007. The entire contents of all the above documents are hereby incorporated by reference.

This invention was made with Government support under Grant No. CHE-0450022 awarded by the National Science Foundation. The Government has certain rights in this invention

### BACKGROUND

1. Field of Invention

This application relates to processes and systems for making particles, and more particularly processes and systems for making particles having a dimension less than about 1 mm.

2. Discussion of Related Art

The contents of all references, including articles, published patent applications and patents referred to anywhere in this specification are hereby incorporated by reference.

An important emerging class of non-spherical colloidal materials are microscopic and nanoscopic particles that have designed shapes and are created by lithographic means (see e.g. Hernandez, C. J.; Mason, T. G. Colloidal alphabet soup: Monodisperse dispersions of shape-designed LithoParticles. J. Phys. Chem. C 2007, 111, 4477-4480). (These will also be referred to as LithoParticles in this specification.) Optical pattern replicating systems, such as high-fidelity lens-based steppers (Madou, M. J. Fundamentals of microfabrication: The science of miniaturization. 2nd ed.; CRC Press: Boca Raton, 2002), typically used to print electronic structures on computer chips, have been used to mass-produce LithoParticles and create Brownian dispersions of an entire particulate alphabet: “Colloidal Alphabet Soup”(Hernandez, C. J.; Mason, T. G. Colloidal alphabet soup: Monodisperse dispersions of shape-designed LithoParticles. J. Phys. Chem. C 2007, 111, 4477-4480). In the basic implementation of this approach, a polymer resist layer can be cross-linked by the optical exposure and, after development, the polymer resist particles can be lifted off of the substrate. This optical approach for making LithoParticles has important and non-obvious differences from earlier approaches (Higurashi, E.; Ukita, H.; Tanaka, H.; Ohguchi, O. Optically induced rotation of anisotropic micro-objects fabricated by surface micromachining. Appl. Phys. Lett. 1994, 64, 2209-2210; Brown, A. B. D.; Smith, C. G.; Rennie, A. R. Fabricating colloidal particles with photolithography and their interactions at an air-water interface. Phys. Rev. E 2000, 62, 951-960; Sullivan, M.; Zhao, K.; Harrison, C.; Austin, R. H.; Megens, M.; Hollingsworth, A.; Russel, W. B.; Cheng, Z.; Mason, T. G.; Chaikin, P. M. Control of colloids with gravity, temperature gradients, and electric fields. J. Phys. Condens. Matter 2003, 15, S11 -S18) that required etching as part of the procedure. Although robotically automated optical exposure can be used to create significant quantities of monodisperse LithoParticles, expensive lithography exposure systems must be continuously used to optically pattern films during the particle production process. Due to the limited availability and expense of these precise optical exposure systems, there would be advantages to other LithoParticle production methods that could rapidly produce shape-designed particles without relying on such optical equipment during the repetitive production process.

Mechanical imprinting, whether thermal or step-and-flash, is a technology that involves bringing two solid plates into contact after depositing a desired material between them (Madou, M. J. Fundamentals of microfabrication: The science of miniaturization. 2nd ed.; CRC Press: Boca Raton, 2002; Chou, S. Y. Nanoimprint lithography and lithographically induced self assembly. MRS Bulletin 2001, 26, 512; Chou, S. Y.; Krauss, P. R.; Renstrom, P. J. Nanoimprint lithography. J. Vacuum Sci. Tech. B 1996, 14 (6), 4129-4133; Resnick, D. J.; Mancini, D.; Dauksher, W. J.; Nordquist, K.; Bailey, T. C.; Johnson, S.; Sreenivasan, S. V.; Ekerdt, J. G.; Willson, C. G. Improved step and flash imprint lithography templates for nanofabrication. Microelectronic Engineering 2003, 69, 412-419). Once the surfaces of the two plates touch, the material only fills trenches or wells in one plate that has been prepared with the desired patterns. Imprinting essentially forces a desired material into voids that have been created in one of the surfaces to form a mold. While the two plates are touching (or nearly touching), a process, such as cross-linking in the case of polymers, can be used to rigidify the material in the mold, and then the plates are separated. During the separation, if the release of the desired material from the corrugated surface can be made efficiently, then the result is a set of raised structures of the desired material on the flat surface of the other plate. Imprinting is a subset of the more general process of embossing, in which a mold is pressed into the surface of a material that is not as rigid and then removed to create raised corrugations that reflect the mold. However, by contrast to embossing, mechanical imprinting involves squeezing out material between two solid plates where they touch, so that only the negative relief corrugations in one plate become filled with the desired material.

Performing mechanical imprinting reproducibly in a production setting can be problematic for many reasons. It is often difficult to achieve good mechanical contact between the two plates over large surface areas. To mitigate this, large sections of the plates are often cut away so that only small, disconnected pedestals containing the desired patterns touch the flat plate. Using pedestals decreases the surface area and production rate significantly. Defects in the surfaces of the plates, dust, or enhanced surface roughness due to wear can preclude the exact contact of the plates, especially for larger substrate sizes. For very small shapes, the wetting properties of the material to be imprinted with the plates can play an important role in determining the success and reproducibility of the imprinting procedure. These are some of the primary reasons why mechanical imprinting has not been widely adopted by the electronics industry as a replacement to more reliable optical approaches. Although imprinting is making some inroads into certain specialty electronics applications, it is uncertain if mechanical imprinting technology will advance to a degree of robustness necessary to overtake existing optical methods in the current race to the sub-50 nm level. Although it is possible to create LithoParticles using imprinting methods, as we and others (Rolland, J. P.; Maynor, B. W.; Euliss, L. E.; Exner, A. E.; Denison, G. M.; DeSimone, J. M. Direct fabrication of monodisperse shape-specific nanobiomaterials through imprinting exists (J. Am. Chem. Soc. 2005, 127, 10096-10100), yet developing alternative approaches for rapidly mass-producing LithoParticles that do not involve imprinting or repetitious exposure by an optical lithography system would be highly useful.

### SUMMARY

A method of producing at least one of microscopic and submicroscopic particles according to some embodiments of the current invention includes providing a template comprising a plurality of discrete surface portions, each discrete surface portion having a surface geometry selected to impart a desired geometrical property to a particle while being produced; depositing a constituent material of the at least one of microscopic and submicroscopic particles being produced onto the plurality of discrete surface portions of the template to form at least portions of the particles; separating the at least one of microscopic and submicroscopic particles comprising the constituent material from the template into a fluid material, the particles being separate from each other at respective discrete surface portions of the template; and processing the template for subsequent use in producing additional at least one of microscopic and submicroscopic particles. The method of producing at least one of microscopic and submicroscopic particles according to an embodiment of the current invention is free of bringing a solid structure, other than the constituent material, into contact with the template proximate the plurality of discrete surface portions during the producing, and is free of bringing the solid structure into contact with the constituent material during the producing.

A multi-component composition according to some embodiments of the current invention includes a first material component in which particles can be dispersed, and a plurality of particles dispersed in the first material component. The plurality of particles is produced by methods according to embodiments of the current invention.

A system for manufacturing at least one of microscopic and submicroscopic particles according to some embodiments of the current invention includes a template cleaning and preparation system; a deposition system arranged proximate the template cleaning and preparation system to be able to receive a template from the template cleaning and preparation system upon which material will be deposited to produce the particles; and a particle removal system arranged proximate the deposition system to be able to receive a template from the deposition system after material has been deposited on the template. The system for manufacturing particles is free of a structural component, other than the constituent material, for contacting with the template proximate a plurality of discrete surface portions of the template, and is free of a structural component, other than the constituent material, for contacting with the constituent material during the producing.

### BRIEF DESCRIPTION OF THE DRAWINGS

The invention is better understood by reading the following detailed description with reference to the accompanying figures in which:

FIG. 1 is a schematic illustration of a repeatable process for making LithoParticles using permanent Well-Deposition Particle Templating (W-DePT) according to an embodiment of the current invention. Starting with the well-template (top), a sacrificial release layer is deposited, then the target particle material is deposited, and the particles in the bottoms of the wells are released by immersion and agitation in a fluid, which causes the sacrificial layer to dissolve (bottom). The LithoParticles are retained in the fluid, and the well-template is cleaned and re-used. Optionally, the patterned film containing holes in the shapes of the particles can be retained for use and/or recycling.

FIG. 2(a) is a schematic illustration of a method of producing a well-template suitable for W-DePT according to an embodiment of the current invention. An SiO₂ layer is deposited on a flat solid Si substrate and is then spin-coated with a photoresist layer. This top resist layer is exposed using an optical lithography system. The exposed resist is developed, yielding a continuous resist pattern that contains holes that reflect the desired particle shapes. Reactive ion etching of the exposed SiO₂ regions then exposes similarly shaped regions of the Si surface. Subsequent chlorine etching to the desired depth creates impressions of the desired well patterns in the Si substrate, and the residual photoresist and SiO₂ are stripped and removed.

FIG. 2(b) shows an SEM image of wells that have the desired square-cross shape that have been etched into a silicon wafer using the method of FIG. 2(a).

FIGS. 3(a)-3(c) show optical micrographs of several stages of the process described in FIG. 1. FIG. 3(a) shows a reflection micrograph of the etched Si well-template showing a high density of wells shaped in the form of square crosses. FIG. 3(b) shows a reflection micrograph after depositing a 100 nm sacrificial release layer of water-soluble Omnicoat and after sputtering 70 nm gold onto the release-treated well-template. FIG. 3(c) shows a transmission micrograph of gold particles after fluid assisted release out of the wells into an aqueous solution.

FIGS. 4(a)-4(b) show number-weighted size distributions of square crosses produced by W-DePT, as measured using SEM images of fifty particles. FIG. 4(a) shows the distribution of the arm width, N(w), measured at the center of an arm, yields an average arm width <w>=1.37+0.04 μm. FIG. 4(b) shows the distribution of the total cross length, N(l), measured from the center of the end of one arm to the center of the end of the opposite arm, yields an average length <1>=4.35+0.06 μm.

FIG. 5 is a schematic illustration of an example of a continuous automated track production system for making LithoParticles using W-DePT according to an embodiment of the current invention. A sacrificial layer is deposited onto a clean well-template, the particle material is deposited, the well-template is brought in contact with a fluid and agitated to release the desired LithoParticles into the fluid, and the well-template is cleaned and dried, ready for the next cycle. Optionally, a continuous patterned film can be collected, separated, and potentially deposited on a flat substrate to produce an optical mask. All devices can be simultaneously operating using multiple well-templates, and a robotic system can transfer the treated well-templates between devices.

FIG. 6 is a schematic illustration of Well-Deposition Particle Templating using a permanent release layer that coats the well-template's surface according to an embodiment of the current invention. The desired particle material is uniformly deposited onto the well-template in a direction perpendicular to the surface of the template. The deposited material does not adhere to the permanent release layer, so simple fluid agitation releases the particles without disturbing the release layer. The particles are separated and retained. Optionally, a film replica containing holes of the desired particle shapes can also be recovered. The well-template is then re-used, and the process is repeated.

FIG. 7 is a schematic illustration of Well-Deposition Particle Templating through solidification of deposited materials according to an embodiment of the current invention. In this example, a permanent release coating has been initially applied to the well-template. Through deposition, the wells are filled with a material that can be solidified; a continuous surface layer may exist. This surface layer is removed by spin-coating or mechanical displacement. The particle material is solidified, and the particles are removed, separated, and retained through fluid-assisted lift-off. The well-template is then re-used and the process is repeated.

FIG. 8 is a schematic illustration of Well-Deposition Particle Templating according to an embodiment of the current invention using a solid well-template with overhanging side-walls. The process is essentially the same as that described for FIG. 1; directional deposition of the particle material normal to the template's surface creates islands of the desired particle shapes inside the wells. These islands do not touch the side-walls, so particle release is very efficient. It is not necessary to coat the side-walls of the wells under the overhang for this process to be successful. However, the bottoms of the wells must be coated with the release material.

FIG. 9 is a schematic illustration to show that Well-Deposition Particle Templating according to an embodiment of the current invention may not work properly when a continuous layer of the particle material is formed over all of the corrugated surfaces. In this example, the well-template has been etched to create wells that have underhanging side-walls. Because these side-walls can accumulate the deposited particle material, even if directionally deposited normal to the template, separated regions of deposited particle material cannot be formed, and no discrete particles can be created or released without removing the top continuous film by a process such as abrasion or polishing.

FIG. 10 is a schematic illustration of Well-Deposition Particle Templating according to an embodiment of the current invention to create non-slab-shaped pyramid shell particles using a template that has wells coated with a permanent release agent. This method resembles that of FIG. 1, but the bottom of the well-template has been patterned to provide a surface that is not completely flat. The well-template can be re-used and this process can be repeated.

FIG. 11 is a schematic illustration of Well-Deposition Particle Templating according to an embodiment of the current invention to create non-slab-shaped solid pyramid particles using a template that has wells with underhanging side-walls. This method resembles that of FIG. 1, but there is an additional step of removing the continuous layer of particle material on the top contiguous surface of the well-template prior to fluid-assisted removal of the discrete particle shapes. As a result, no continuous film is created in this process. The well-template can be re-used and this process can be repeated.

FIG. 12 is a schematic illustration of a repeatable process for making LithoParticles using permanent Pillar-Deposition Particle Templating (P-DePT) according to an embodiment of the current invention. Starting with the pillar template (top), a sacrificial release layer is deposited, then the target material for the particle is deposited, and the particles at the tops of the pillars are released by immersion into a fluid and dissolution of the sacrificial layer (bottom). The LithoParticles are retained in the fluid (arrows at left bottom), and the pillar template is cleaned and re-used (arrows at right).

FIG. 13(a) is a schematic illustration of a method of producing a pillar template suitable for P-DePT according to an embodiment of the current invention. A flat solid substrate is coated with a resist layer; this resist layer is exposed using a lithography system, the exposed resist is developed and descummed, yielding resist islands that reflect the desired particle shape; the exposed substrate is etched, the residual photoresist is stripped away, and the etched substrate is cleaned.

FIG. 13(b) shows a scanning electron micrograph of a pillar-template for making a plurality of plate-like particles that resemble square crosses. This template is made by ion etching a silicon surface according to an embodiment of the current invention.

FIGS. 14(a)-14(d) show reflection optical micrographs for examples according to an embodiment of the current invention. FIG. 14(a) shows a 45 nm thick gold layer that has been deposited on the tops of the silicon square cross pillar-template by sputtering. Below the gold layer is a 20 nm coating of a sacrificial polymeric release agent, OMNICOAT. FIG. 14(b) shows fluid-assisted release of particles: the pillar-template is immersed in water and agitated to increase the rate of dissolution of the release layer. FIG. 14(c) shows the pillar-template can then be re-used. FIG. 14(d) shows liberated cross-shaped gold particles are separated and recovered in aqueous solution (optical transmission micrograph).

FIG. 15 is a schematic illustration of an example of a continuous automated track production system for making LithoParticles using P-DePT according to an embodiment of the current invention. Clean pillar templates are introduced (top), and adhesion promoter is applied, the sacrificial layer is deposited, the particle layer is deposited, the pillar template is brought in contact with a fluid and agitated to release the desired LithoParticles into the fluid, and the wafer is cleaned and dried (bottom), ready for the next cycle. All devices can be simultaneously operating using multiple templates, and a robotic system transfers the pillar templates between devices (arrows).

FIG. 16 is a schematic illustration of P-DePT of complex non-slab particle shapes using a permanent release coating according to an embodiment of the current invention. The tops of the pillars, which were originally flat, have been etched to provide a structured surface and then permanently coated with a release agent (top of FIG. 16). As shown here, the top surface of the pillars may even be etched to provide negative relief patterns, such as pyramidal or conical depressions. A desired particle material (bottom of FIG. 16) is deposited onto the surface, and fluid agitation releases the particles from the pillars. The particles are retained in the fluid and the pillar template can be re-used.

FIG. 17 is a schematic illustration of P-DePT of complex non-planar particle shapes having uniform thickness using a permanent release coating. The tops of the pillars, which were originally flat, have been etched to provide a structured surface (e.g. a pointed pyramid or cone) and permanently coated with a release agent (top of FIG. 17). The desired particle material (bottom of FIG. 17) is deposited onto the release-coated sculpted pillar surfaces using a directional process that creates a layer having uniform thickness, and fluid agitation releases the non-planar pyramidal particles from the pillars. These non-planar LithoParticles are retained in the fluid, and the pillar template is re-used.

FIG. 18 is a schematic illustration of another embodiment of P-DePT according to the current invention.

FIG. 19 is a schematic illustration of another embodiment of P-DePT according to the current invention.

FIG. 20 is a schematic illustration of another embodiment of P-DePT according to the current invention.

FIG. 21 is a schematic illustration of another embodiment of P-DePT according to the current invention.

FIG. 22 is a schematic illustration of another embodiment of P-DePT according to the current invention.

FIG. 23 is a schematic illustration of another embodiment of P-DePT according to the current invention.

FIG. 24 is a schematic illustration of another embodiment of P-DePT according to the current invention.

FIG. 25 is a schematic illustration of an example of a complex relief pattern according to an embodiment of the current invention. When reproduced over the entire surface of a template, such a pattern can be used to produce plate-like particles in the shape of square slabs with up to 100% area coverage and efficiency. The cutaway view shown here is just a portion of the template surface that shows how the different square levels can be configured with neighboring levels so that isolated square particles are produced over the entire surface of the template. This template has been constructed to provide multiple levels of relief, not just simple pillars or wells. In this example, there are six different relief levels, and directional deposition of the desired particle material from above (from top of the page toward the bottom) onto the square-shaped surfaces will produce square-shaped particles from all six different relief levels. A single release step could be used to release particles from all six levels into solution. This would be an efficient way of liberating particles from all of the surfaces at different relief levels. Alternatively, multiple release steps could be used to release particles from each of the six different levels of the template.

FIG. 26 is a schematic illustration of post-release attachment of one material onto LithoParticles' surfaces.

FIG. 27 is a schematic illustration of post-release attachment of one or more materials onto LithoParticles' surfaces.

FIG. 28 is a schematic illustration of post-release attachment of one or more materials onto LithoParticles' surfaces utilizing a permanent release layer.

FIG. 29 is a schematic illustration of post-release attachment of one material onto LithoParticles' surfaces.

FIG. 30 is a schematic illustration of post-release attachment of one or more materials onto LithoParticles' surfaces.

FIG. 31 is a schematic illustration of pre-release attachment of one material onto a portion of LithoParticles' surfaces.

FIG. 32 is a schematic illustration of pre-release and post-release attachment of two different materials onto specific portions of LithoParticles' surfaces.

FIG. 33 is a schematic illustration of post-release attachment of two different materials onto site-specific portions of bilayer LithoParticles' surfaces.

FIG. 34 is a schematic illustration of pre-release and post-release attachment of three different materials onto site-specific portions of bilayer LithoParticles' surfaces.

FIG. 35 is a schematic illustration of complex hybrid bilayer LithoParticles made using two relief levels on the top pillar surfaces.

FIG. 36 is a schematic illustration of pre-release attachment of two different materials onto site-specific portions of bilayer LithoParticles' surfaces.

FIG. 37 is a schematic illustration of pre-release and post-release attachment of four different materials onto site-specific portions of surfaces of LithoParticles comprised of two particle materials.

FIG. 38 is a schematic illustration of site-selective modification of a portion of the surfaces of LithoParticles made using well-deposition templating.

FIG. 39 is a schematic illustration for incorporating an internal layer of microscale or nanoscale particulates into LithoParticles.

FIG. 40 is a schematic illustration for incorporating an external layer of microscale or nanoscale particulates on exposed surfaces of LithoParticles prior to release.

FIG. 41 is a schematic illustration for incorporating an external layer of microscale or nanoscale particulates on exposed surfaces of LithoParticles after release.

FIG. 42 is a schematic illustration of pre-release shape modification of LithoParticles: rounding sharp edges and corners.

FIG. 43 is a schematic illustration of post-release shape modification of LithoParticles: rounding sharp edges and corners.

FIG. 44 is a schematic illustration of post-release size and shape modification of LithoParticles: coating LithoParticles in fluid with a second type of particle material.

FIG. 45 is a schematic illustration of pre-release size and shape modification of LithoParticles: coating LithoParticles with a second type of particle material.

FIG. 46 is a schematic illustration of one exemplary embodiment for directed assembly of surface-modified particles.

FIG. 47 is a schematic illustration of one exemplary embodiment for directed assembly of surface-modified particles.

FIG. 48 is a schematic illustration of one exemplary embodiment for directed assembly of surface-modified particles.

FIG. 49 is a schematic illustration of one exemplary embodiment for directed assembly of surface-modified particles.

### DETAILED DESCRIPTION

In describing embodiments of the present invention illustrated in the drawings, specific terminology is employed for the sake of clarity. However, the invention is not intended to be limited to the specific terminology so selected. It is to be understood that each specific element includes all technical equivalents which operate in a similar manner to accomplish a similar purpose.

Some embodiments of the current invention provide methods for producing microscopic and/or submicroscopic particles. The methods according to some embodiments of the current invention include providing a template that has a plurality of discrete surface portions, each discrete surface portion having a surface geometry selected to impart a desired geometrical property to a particle while being produced. Each of the discrete surface portions can be, but are not limited to, a flat surface, a curved surface, a complex contoured surface, a surface with a plurality of subsurface regions, or any combination thereof. Herein, microscopic refers to the range of length scales equal to and greater than one micrometer, including length scales ranging up to about one millimeter. Herein, submicroscopic refers to the range of length scales below one micrometer, including length scales ranging down to about one nanometer.

The methods according to some embodiments of the current invention also include depositing a constituent material of said at least one of microscopic and submicroscopic particles being produced onto said plurality of discrete surface portions of said template to form at least portions of said particles. The constituent material is a material in the composition of the particles being manufactured. The broad concepts of the current invention are not limited to any specific constituent materials. There is an extremely broad range of materials including organic, inorganic, composite, multi-component and any combination thereof that could be used in various embodiments of the current invention. The depositing can be a directional deposition in some embodiments of the current invention that, for example, leaves at least a fraction of wall portions around the discrete surface portions uncoated by the constituent material. The depositing can include spin-coating, spray-coating, dip-coating, sputtering, chemical vapor deposition, molecular beam epitaxy, electron-beam metal deposition, or any combination thereof in some embodiments of the current invention.

The methods according to some embodiments of the current invention further include separating at least one particle from the template in which the particle separated has the constituent material in its composition. The particle may be separated into a fluid, for example, into a liquid in some embodiments of the current invention. In some embodiments there may be one or a small number of particles separated from the template, but in other embodiments, there can be a very large number of particles separated in the same separation step. For example, in some embodiments there could be hundreds of thousands, millions and even billions or more particles separated from the template in the same step.

The methods according to some embodiments of the current invention further include processing the template for subsequent use in producing additional particles. Once the template is processed for subsequent use, the above-noted depositing and separating steps can be repeated to produce additional particles. The template may be reprocessed many times according to some embodiments of the invention to mass produce, in assembly-line fashion, very large numbers of the particles. The method of producing particles according to such embodiments of the current invention does not include pressing a structural component against the template to control the application of material to the template, such as is done with printing methods.

### Well-Deposition Particle Templating

An embodiment of the current invention is a process which will be referred to as “Well-Deposition Particle Templating” (W-DePT). W-DePT involves only a single patterned solid plate and an appropriate deposition and release scheme. A solid “well-template” is created by permanently etching a solid surface to make one or more wells that reflect the desired shape or shapes. Although optical or electron beam (e-beam) lithography is typically used in combination with etching to first make this “well-template”, the remaining steps that are repeated for mass-producing particles do not require any exposure or etching systems.

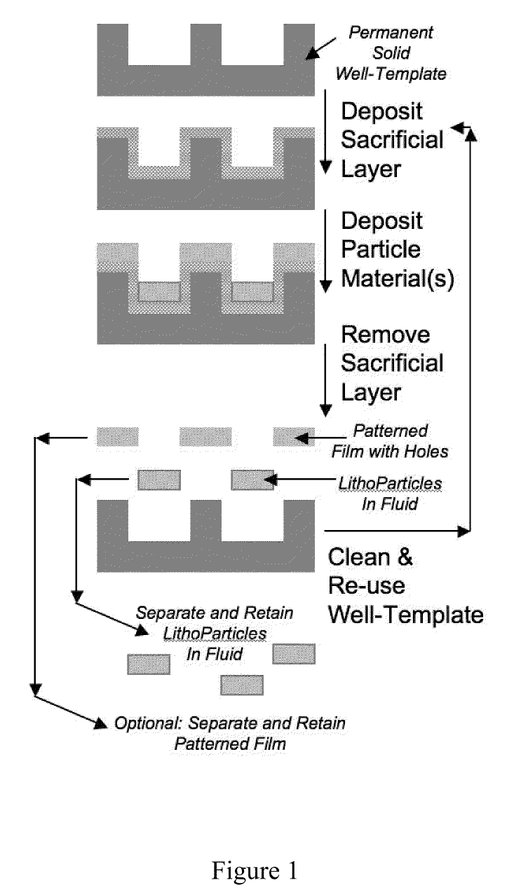

In a simple implementation, W-DePT can be achieved by: (1) depositing a thin layer of a release agent, such as a temporary sacrificial release layer (e.g. fluid-soluble polymer) or a permanent molecular coating (e.g. fluorinated siloxane chains) over the corrugated surface of the well-template; (2) depositing the desired particle materials at a desired thickness through various deposition processes, such as sputtering, physical vapor deposition (PVD), chemical vapor deposition (CVD), or spin-coating; and then (3) releasing the particles from the wells into a fluid, usually using some form of agitation (See FIG. 1). Fluid-assisted release can involve dissolving a temporary sacrificial release layer, or it can simply dislodge particles from a surface that may be coated with a permanent release agent. Since the well-template is not altered by the deposition and release processes, it can be re-used, and the templating process can be rapidly repeated. We have used W-DePT to mass-produce particles having less than 5% polydispersity in thickness and linear cross-sectional dimensions with an efficient release rate exceeding 99%. By performing W-DePT using multiple templates simultaneously, LithoParticle production rates can be made very high without the difficulties and added complexities of imprinting methods involving mechanical contact of a flat plate with a patterned surface. Moreover, repeated patterned optical exposure is also not necessary to achieve a high-throughput production scheme.

### W-DePT Examples

### Methods for Producing a Well-Template

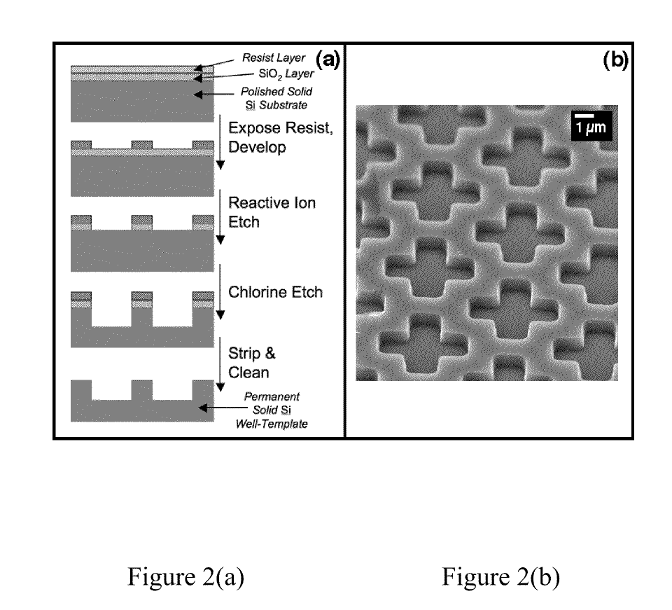

Many lithographic methods can be used to create a patterned “well-template” suitable for W-DePT. As an example, we describe one approach that can be used to create a well-template for making cross-shaped particles with W-DePT. This process is shown schematically in FIG. 2(a). A densely populated optical reticle-mask (not shown) of chrome on quartz that contains patterns of many disconnected cross-shapes is designed and produced using e-beam lithography following standard methods (Madou, M. J. Fundamentals of microfabrication: The science of miniaturization. 2nd ed.; CRC Press: Boca Raton, 2002). This optical reticle-mask is not required for producing a well-template, but it provides a convenient means of more easily producing more than one well-template from an optical, rather than an e-beam, process. If only one template is desired or if the desired resolution lies below the optical limit, an e-beam exposure system could be used to directly pattern a resist layer, and subsequent etching could provide the well-template without any need for an optical reticle-mask. Assuming that the optical reticle-mask has been produced, a flat polished silicon wafer is coated with 170 nm of silicon dioxide using plasma-enhanced CVD and then a 1.6 micron layer of polymer photoresist (Shipley AZ-5214) using a spin-coater at 3,000 RPM. A mercury i-line projection stepper system (Ultratech XLS-2145i), exposes the resist-coated wafer with patterned ultraviolet light that has passed through the reticle-mask. After normal development, the crosslinked resist forms an interconnected layer that contains many voids in the form of square crosses. Inside these voids, the silicon dioxide layer is exposed. A reactive ion etcher (RIE) is used to completely etch through the oxide layer, revealing the silicon surface. This exposed silicon surface is permanently etched using a chlorine etcher to a depth of 0.8 microns, creating many wells in the shapes of crosses. The residual protective resist and remaining oxide are then stripped (i.e. removed) from the silicon surface using piranha (a mixture of 70% sulfuric acid and 30% hydrogen peroxide) and an aqueous solution of HF (50%). Depending upon the desired particle size and shape, the resulting well-template on a five-inch silicon wafer can contain up to one billion or more wells (i.e. negative relief features) that define the desired particle shapes in negative relief, shown in the scanning electron micrograph of FIG. 2(b). The area fraction of the wells defining the desired shapes can be low, although there is an advantage to having a higher density for particle production throughput, provided the wells remain discrete and do not interconnect.

Choosing appropriate etching conditions and rates is important in some embodiments in order to obtain uniform side-walls without undesirable defects, such as pronounced scalloping, that could inhibit release. Furthermore, the etch depth has been made larger than the maximum desired thickness of the particles. Extremely high etch depths of many microns may not be desirable in some embodiments since deeper wells can reduce the rate and efficiency of release of particles that are formed in them. The basic requirement for the template according to this embodiment of the invention is that it is a solid material containing a permanent patterned structure of wells that define desired particle shapes. Usually, polished solid materials, such as silicon or quartz wafers, represent the easiest candidates for patterning at length scales less than ten microns for making colloidal particles. However, materials other than silicon and quartz can be used for the well-template.

A wide variety of lithographic approaches other than the one we have described can be used to produce the patterned “well-template”. These approaches may not involve depositing a silicon oxide layer onto a silicon wafer, performing resist-based optical lithography to print the repeating disconnected patterns of particle shapes, nor etching silicon dioxide, as we have described in our example. The key characteristic of a well-template according to this embodiment of the invention is essentially a solid material that has at least one surface that has been permanently patterned to have one or more wells of a desired shape into which at least the desired particle material can be deposited.

Mass-Producing Particles using Well-Deposition Templating

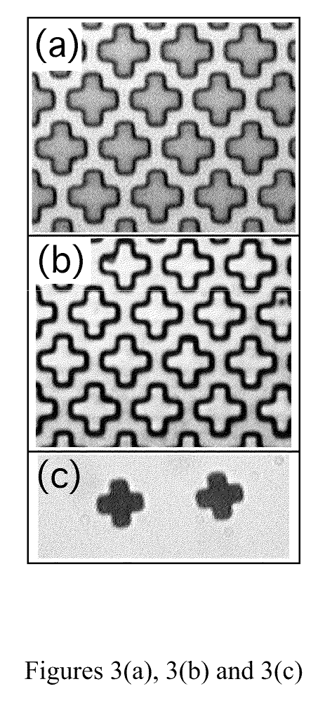

Once the well-template has been made, LithoParticles can be mass-produced by a succession of steps that involve deposition and fluid-assisted release. As an example, using the well-template of square-crosses, we produce an aqueous suspension of cross-shaped gold particles by the process outlined in FIG. 1. We coat all of the surfaces, including the side-walls, of the well-template with a release agent. This could be a simple permanent molecular layer, such as a fluorocarbon, that provides low surface energy contact with the desired material for the particles, or it could be a layer of deposited sacrificial material (e.g. a water-soluble polymer) that can be removed in a subsequent release step. For our example here, we use the second alternative. When necessary, we treat the well-template with an adhesion promoter, hexamethyldisilazane (HMDS), in order to facilitate the process of uniformly coating of the sacrificial material over all surfaces of the patterned well-template, including the side-walls. For example, we create a thin water-soluble sacrificial release layer (e.g. Omnicoat) over the surface of the well-template by spray-coating using an atomizer (e.g. air-brush), spinning the wafer at 3,000 RPM to remove any excess polymer solution that remains on the top surface of the wafer. Baking at 200° C. for one minute evaporates the solvent for the release agent, leaving behind a thin solid layer that uniformly coats all surfaces of the well-template to a thickness of approximately 100 nm. Next, we uniformly deposit the desired thickness, 70 nm, of the desired particle material, gold, onto the coated well-template using sputtering. Although this deposition also coats the top surface of the template, not just the wells, the top surface layer is completely interconnected over macroscopic length scales, so there are no small particles that would be formed from this top layer of deposited material. After depositing the desired particle material, the well-template is immersed in Omnicoat developer (2.28% tetramethyl ammonium hydroxide), and agitated in the developer using an ultrasonic bath to cause the sacrificial layer to rapidly dissolve and the gold particles to be released into solution, as shown in FIG. 3(c). The time necessary to release the particles from the wells is typically about two minutes. Care must be taken not to make the ultrasonic agitation too severe; otherwise, particles can be broken by the agitation.

As a by-product of the W-DePT process, a large interconnected film of the desired particle material is created. In the example given above, a layer of patterned gold with cross-shaped holes is also created and lifted off into solution at the same time as the particles. In principle, the intact patterned film could be used to create an optical mask by deposition onto a quartz surface or for shape-specific filtration if mounted on an appropriate porous substrate. Because this film is much larger than the particles that are produced, it can be easily separated from the particles during or after the fluid-assisted release process. If the particle material is valuable and a continuous film is not a desired product, then this interconnected layer can be recovered and potentially recycled. In practice, thin continuous films can be very fragile, and more vigorous agitation used to release particles can potentially tear or break them into smaller pieces. As a result, mild agitation that does not lead to release of the particles can be used to recover an intact film after lift-off, and subsequent stronger agitation can be used to release the particles.

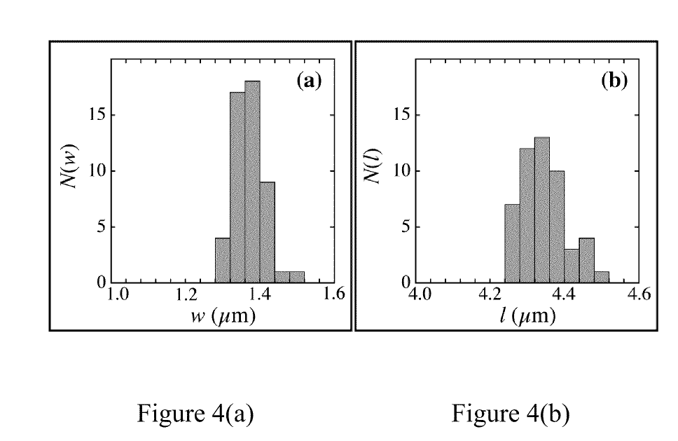

Scanning electron microscopy (SEM) images reveal that the number-weighted polydispersity of the arm lengths and thicknesses of the crosses to less than 5%. In FIGS. 4(a) and 4(b), we show the size distributions N(w) and N(l) corresponding to the width, w, of the arms of the crosses (measured at the middle of the arm) and the total end-to-end length, l, of the arms of the crosses, respectively. We find that the number-weighted average width is <w>=1.37+0.04 μm and the average total length is <l>=4.35+0.06 μm, where uncertainties correspond to the standard deviations of the respective distributions. The polydispersity of the thickness, t, is more difficult to measure for thin particles that tend to deposit flat onto the conducting surface, and we estimate the average thickness to be approximately <t>≈70 nm. Based on uniformity of coatings sputtered on flat surfaces, we estimate the uncertainty in the thickness of the ensemble to be about 5 nm -10 nm. More precise deposition devices that spin and rotate the substrate while they deposit, such as those used to create thin coatings on optical lithography masks, can provide a higher degree of uniformity in thickness over a larger surface area.

The polydispersity of the edge lengths is essentially set by the precision of the well-template (i.e. through the exposure and etching processes), whereas the polydispersity of the thickness by the uniformity of the deposition process for coating the wells with the desired materials. For the example W-DePT implementation that we have described using a polymer release layer and gold, the surface roughness of the top and bottom flat layers of the particles is determined by the roughness of the deposited polymer layer and the uniformity of the sputtering process. We have performed W-DePT using the same template repeatedly without any noticeable degradation of the well-template or deterioration of the particle uniformity. Occasionally, the surfaces of the silicon well-template can be non-destructively cleaned using piranha and HF solutions to ensure maximum fidelity. For the method we have described to make gold crosses, using optical reflection microscopy, we estimate the efficiency of release to be greater than 99% after agitating for less than two minutes using an ultrasonic bath, with less than one particle in a thousand remaining stuck in a well. Non-directional vapor deposition of the sacrificial layer, rather than spray-coating and spin-coating, would most likely improve this release efficiency. It is obvious that this approach for making particles will not be successful if the sides of the wells become coated with the particle material, thereby connecting the continuous film on top of the template to the particles in the wells. So, directional deposition methods that do not coat the side-walls, such as sputtering deposition or evaporative deposition normal to the surface or using physical vapor deposition (e.g. thermal or e-beam), offer distinct advantages for the simple example of W-DePT that we have shown. Likewise, W-DePT may not yield discrete particles in its simplest form if the particle layer becomes too thick due to over-deposition, such that the material in the wells would form rigid contacts with the top continuous film.

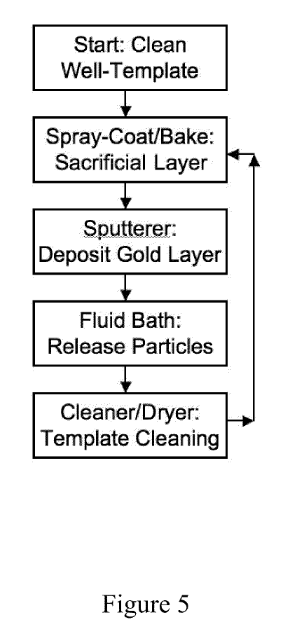

Completely automated W-DePT can be performed in parallel using many templates that are continuously recirculated by a robotic track system. Identical well-templates are circulated into a spray/spin coater, a baker, a sputterer, a fluid agitation bath, a cleaning tank, a drying stage, and then back to the spray/spin coater to complete the loop (see FIG. 5). The spray/spin coater, baker, cleaning tank and/or drying system can be components of a template cleaning and preparation system of a system for manufacturing particles according to an embodiment of the current invention. The sputterer is one example of a possible deposition system for manufacturing particles according to an embodiment of the current invention. The deposition system is not limited to only a sputterer and may include other deposition systems including those described in references to various examples herein. The ultrasonic bath is one example of a possible particle removal system according to an embodiment of the current invention. However, systems for manufacturing particles according to various embodiments of the current invention are not limited to this specific example. At the fluid agitation stage, LithoParticles are collected and retained in a fluid. The track system is only one possible way of performing high-throughput production. A rotary carrousel that provides parallel processing of several identical well-templates could also be used. Alternatively, various operations could be performed on specific regions of a well-template as it is rotated or translated, if these deposition methods can be scaled down. One of the main advantages of the automated parallel W-DePT replication process is that it doesn't require a full-time robotic optical exposure system; this system usually represents the most expensive part of any lithographic fabrication production line.

### Well-Deposition Particle Templating: Permanent Release Layer

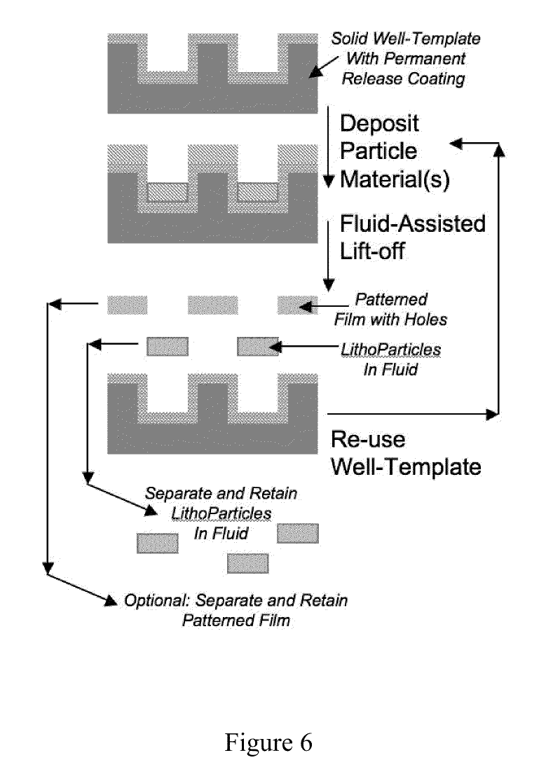

A simple alternative method for making the LithoParticles using W-DePT involves permanently bonding a low-surface energy release agent to the surfaces of the well-template. This release agent can take the form of a fluorocarbon, fluorohydrocarbon, or fluoro-siloxane with appropriate reactive groups for bonding these molecules to the well-template surfaces. This type of low-surface energy coating can be applied using standard methods of surface treatment. After treating the well-template by coating and bonding a high surface density of such molecules to all of the patterned surfaces, the treated well-template surface will have only a very weak attractive interaction with a desired particle material. Once this particle material has been deposited into the wells, the permanent release coating permits facile fluid-assisted release of particles from the wells without the need for the fluid to dissolve a sacrificial release layer. In this variation of W-DePT, shown in FIG. 6, directional deposition normal to the template's surface yields particles in the wells and a continuous film on the top surface. Fluid-assisted release involving agitation can dislodge the particles from the wells and the upper continuous film without any deposition and removal of a sacrificial layer.

### Well-Deposition Particle Templating: Solidification of a Material in the Wells

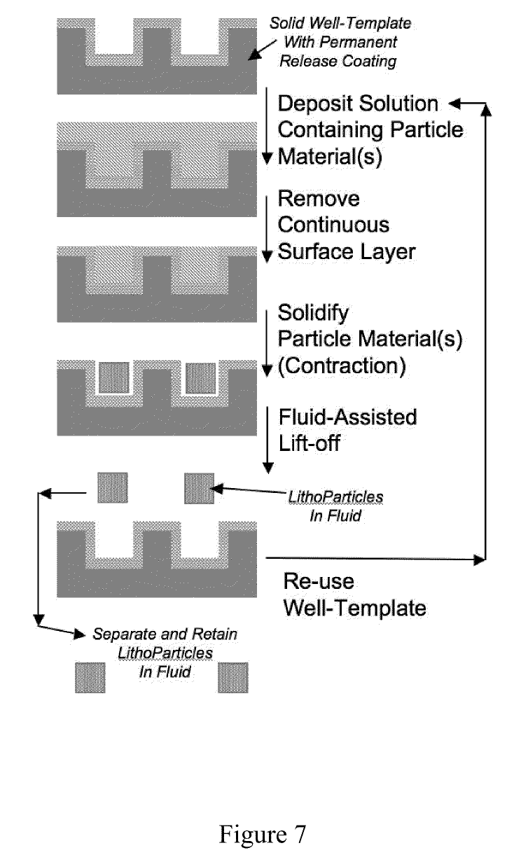

Another interesting variation of W-DePT, which can employ either a temporary or permanent release layer, involves depositing a desired target particle material in a liquid base into the wells and causing a solidification of that target material by some other process, such as aggregation, gelation, phase changes due to temperature or pressure, or evaporation. This process is shown in FIG. 7 for the case of an inorganic silicon dioxide (i.e. silica) xerogel (Himcinschi, C.; Friedrich, M.; Murray, C.; Streiter, I.; Schulz, S. E.; Gessner, T.; Zahn, D. R. T. Characterization of silica xerogel films by variable-angle spectroscopic ellipsometry and infrared spectroscopy. Semicond. Sci. Technol. 2001, 16, 806-811). Deposition of the sol liquid into the wells is achieved by spray coating, and then removing residual liquid from the well-template top surface by spin coating with the pure solvent. Heat treatment causes a porous gel of the silicon dioxide to form in each of the wells, and, by further heat treatment, these gel particles can be made to contract uniformly to form a more dense porous glass which retains the original shape of the wells. The shrunken particles retain the shapes of the well, and the efficiency of recovery for this method can be quite high. Due to the contraction, this method would work well for shapes such as square crosses, but may be problematic for shapes that contain holes. For such toroidal shapes, or donuts, it may be necessary to liberate the gel at an early stage from the wells before applying further heat treatment to shrink the particles outside of the wells.

### Well-Deposition Particle Templating: Overhanging Side-Walls

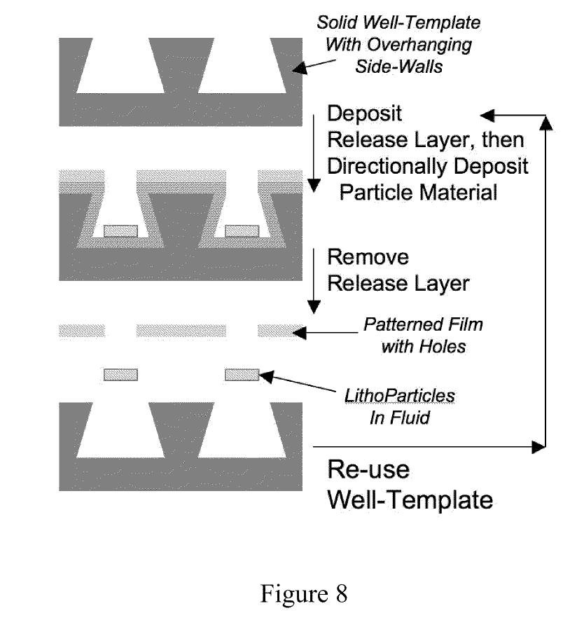

Well-templates that have overhanging side-walls (Madou, M. J. Fundamentals of microfabrication: The science of miniaturization. 2nd ed.; CRC Press: Boca Raton, 2002) can be used for W-DePT, provided directional deposition of the desired target material for the particles is used. For instance, for gold deposition normal to the surface of an overhang well-template, particles can still escape from the wells during the release step, as shown in FIG. 8. In this case where directional deposition is normal to the template surface, it is not necessary for the release material to coat the side-walls of the wells in order for release to be feasible. It is sufficient for the release material to coat just the bottoms of the wells in the region where the particle material is deposited. Because the constriction at the top of the wells with overhanging side-walls is no larger than the deposited particle material, the particles can be released from the wells. Some forms of non-directional deposition into wells that have overhanging side-walls could create particles that are larger than the constriction. This situation could preclude W-DePT, because the constriction at the top of the wells could inhibit the release of the particles, even if they have been successfully liberated from the bottoms of the wells.

### Well-Deposition Particle Templating: Limitations—Underhanging Side-Walls

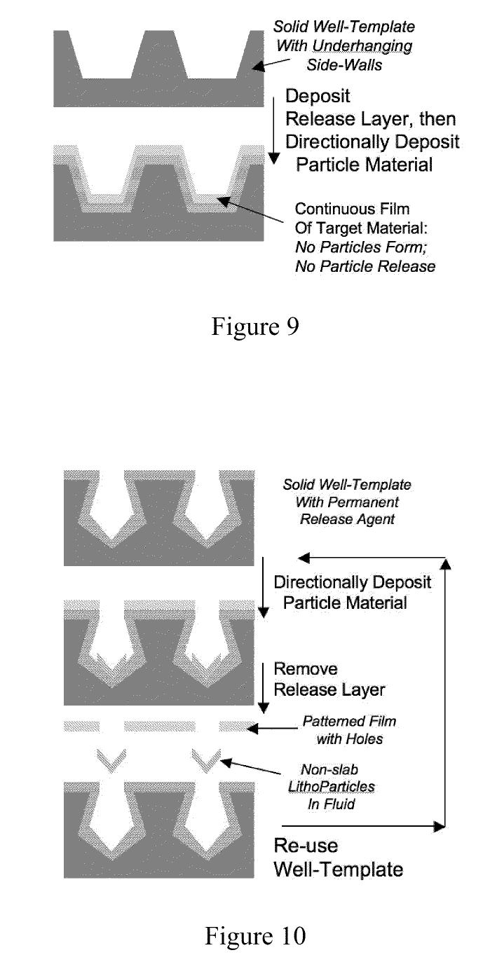

Several situations can lead to difficulties with the efficiency of production and release of particles by basic forms of W-DePT. The simplest W-DePT approaches may not produce well-separated and discrete particles if a well-template has side-walls that are “underhanging”, rather than vertical or overhanging. For instance, deposition of the particle material into wells that have beveled underhanging side-walls, created by anisotropically etching silicon (Powell, O.; Harrison, H. B. Anisotropic etching of {100} and {110} planes in (100) silicon. J. Micromech. Microeng. 2001, 11, 217-220), could simply create a continuous layer of the desired particle material over a sacrificial release layer, as shown in FIG. 9. Regardless of the structure of the side-walls, if the deposition of particle material, whether by directional processes or not, completely caps off and separates a sacrificial release layer underneath the particle layer from the fluid, then fluid-assisted particle release will be precluded. This could also occur if the release material does not adequately coat the side-walls, causing the particle material to touch and potentially stick to the side-walls. Moreover, even if the particle material does not strongly adhere to the side-walls, particle material could also cut off access of the fluid to the release layer underneath the particle material in the wells. This would prevent the fluid from dissolving the sacrificial release layer, so release of the particles could not ensue. Even if the side-walls were coated with the sacrificial release agent, if a very thick layer of particle material is deposited into the wells, the process of dissolving the release material between the side-walls of the wells and the particle material filling the wells would be slow. Flushing or flowing the fluid over the surface of the well-template could speed up the release.

Thus, one of the requirements of the simplest versions of W-DePT is that the deposition onto the well-template should create separate, disconnected regions of the desired particle material in each of the wells. The efficiency and rate of release of the LithoParticles from the wells can depend strongly on the thickness of the sacrificial layer, the side-wall geometry of the wells, and the method of deposition of both the sacrificial and particle layers. If the release layer is very thin on the side-walls, then the convective hydrodynamic penetration of the fluid to dissolve the release layer underneath the particles in the wells can be slow, because the region where it can penetrate is more highly constricted. Ultrasonic agitation can be used to expedite the release process, but even this more extreme form of agitation may fail. The combination of the well-template structure and the deposition steps should be chosen in such a manner as to (1) provide discrete structures of the desired particle material in the wells, (2) ensure that these discrete particle structures can be essentially completely liberated from the wells on the well-template, and (3) preserve the structural fidelity of the well-template so that it can be re-used.

### Well-Deposition Particle Templating: Complex Three-Dimensional Shapes

The bottoms of the wells in the well-template need not be flat, and if they are appropriately shaped by either deposition or etching processes (Powell, O.; Harrison, H. B. Anisotropic etching of {100} and {110} planes in (100) silicon. J. Micromech.

Microeng. 2001, 11, 217-220), it is possible to create particles that have highly complex three-dimensional geometries. In FIG. 10, we show a variation of the basic process in which the bottom of the well-template has been etched to form a complex contour, such as a pyramid-shaped (or conical) well-bottom. In this example, the well-template has been treated with a permanent release agent, although a sacrificial release agent could also be used. Directional deposition of a layer having constant thickness normal to the template surface and fluid-assisted release lifts off shell-like LithoParticles resembling pyramids (or cones) that retain the contours of the well-bottom. Although engineering the well-template will typically be more complex than for simple flat-bottomed wells, once the template has been created, the particles can be mass-produced by repeating only the deposition and release processes.

### Well-Deposition Particle Templating: Remove Deposited Material to Free Particles

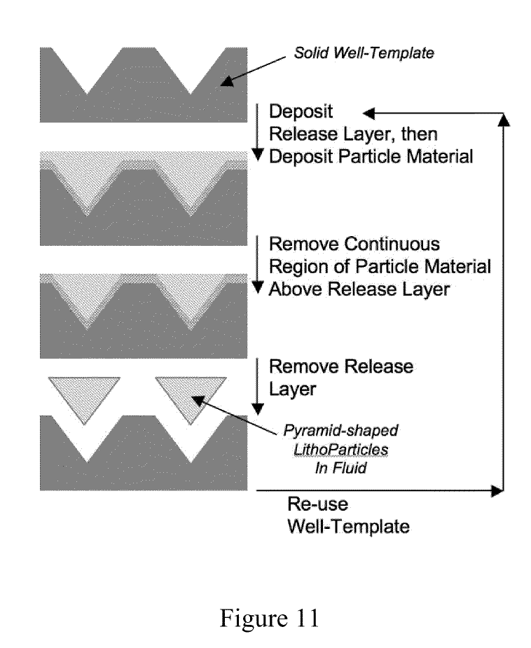

W-DePT can be used to make particles that are not slab-like, even with undercut well-templates, if the top continuous layer of material can be removed by a process without also removing material deposited into the wells. This can be achieved by processes such as, for liquid-borne materials, by spinning off the top continuous layer in a whole surface process reminiscent of edge bead removal of resist at the edges of wafers (Madou, M. J. Fundamentals of microfabrication: The science of miniaturization. 2nd ed.; CRC Press: Boca Raton, 2002). For instance, it would be possible to make particles such as solid pyramids by etching a well-template that has indentations in the form of pyramids, depositing a release layer and then particle materials, spinning off the top surface of the deposited particle layer (thereby creating disconnected islands of particle materials in the wells), solidifying the material in the wells, and releasing the particles from the wells, as we show in FIG. 11.

Many variations of deposition of the sacrificial layer and for the target material layer are possible once the well-template has been made. These materials include organics (e.g. polymers), natural and synthetic biomolecules, inorganics (e.g. conductors, semi-conductors, insulators, including nitrides and oxides), metal-organic frameworks (MOFs)( Roswell, J.; Yaghi, O. M. Effects of functionalization, catenation, and variation of the metal oxide and organic linking units on the low-pressure hydrogen adsorption properties of metal-organic frameworks. J. Am. Chem.

Soc. 2006, 128, 1304-1315), and metals, or combinations of any of these compositions. Particles can be comprised of dense solids, porous solids, flexible solids, or even tenuous gels. LithoParticles made using W-DePT can also contain nanoscopic particulates, such as quantum dots, gold or silver nanoclusters, magnetically-responsive iron oxide, or molecules, such as fluorescent dyes or biologically active drugs. Performing multiple depositions of different desirable target materials prior to the release step can be used to make hybrid bi-layer or multi-layer particles. These deposition methods include, but are not limited to, spin-coating, spray-coating, dip-coating, sputtering, chemical vapor deposition (CVD), molecular beam epitaxy (MBE), and electron-beam metal deposition (EBMD). Release can be made into aqueous or non-aqueous solvents for further chemical surface treatment to increase particle stability against aggregation. Particle release could take place in a wide range of fluids, including supercritical fluids or even gases, not just liquids.

Well-Deposition Particle Templating is considerably different than mechanical imprinting of features including discrete particle shapes. To perform W-DePT, no mechanical lithography device for imprinting, necessary to ensure good mechanical contact between two plates everywhere over the entire surface of the wafer, is needed. Moreover, the performance of W-DePT in reproducibly creating shapes repeatedly from the same template is not nearly as sensitive to dust, wear, and surface imperfections as mechanical imprinting. Instead, to make LithoParticles, only a single patterned substrate, the “well-template”, is required, along with an appropriately chosen deposition and release method. The internal feature sizes and overall dimensions of the particles are not limited to the microscale; direct e-beam writing, x-ray lithography, or deep-UV lithography to a resist-coated surface and subsequent etching could make templates with internal particle features, such as arm widths on the crosses, and overall particle lateral dimensions, smaller than 50 nm.

### Pillar-Deposition Particle Templating

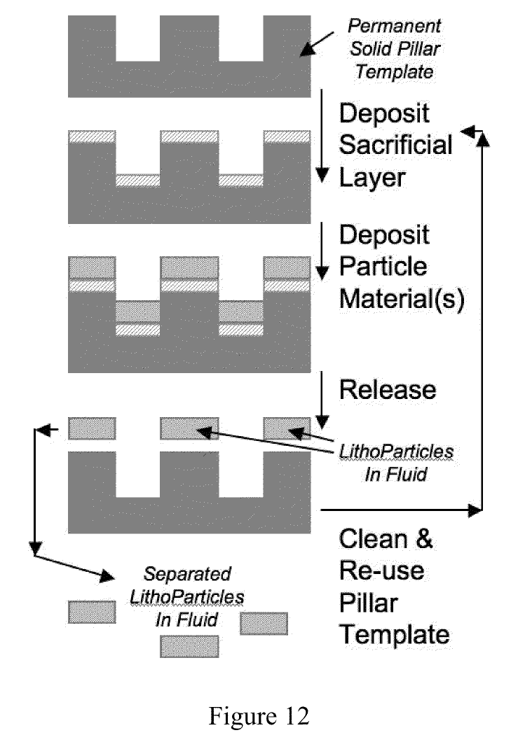

According to another embodiment of the current invention, LithoParticles can be mass-produced from a solid template that has been permanently etched to make pillars that define their cross-sectional shape in a process called “Pillar-Deposition Particle Templating” (P-DePT). Although making the patterned pillar-template, which may contain billions of replicas of a portion of a desired particle shape or different shapes in positive relief, can rely on optical or electron beam (e-beam) lithography, the remaining steps for particle production do not. A simple implementation of P-DePT consists of the following steps: coating the pillars with a thin layer of a release agent, such as a sacrificial layer of water-soluble polymer; depositing the desired particle materials at a desired thickness through various deposition processes, such as sputtering, chemical vapor deposition (CVD), or spin-coating; and then releasing the particles from the pillars into water by dissolving the sacrificial layer using an aqueous solution, as shown in FIG. 12. Since the pillar template can be re-used, the process can be rapidly repeated, and P-DePT is highly effective at producing particles with less than 5% polydispersity in thickness and linear cross-sectional dimensions with a very efficient release rate exceeding 99%. By performing P-DePT using multiple pillar-templates in parallel, it is possible to increase production rates without having to also increase the number of optical exposure systems.

The P-DePT method can facilitate the large-scale production of new kinds of soft multi-phase materials, particularly dispersions of particulates in viscous liquids (Russel, W. B.; Saville, D. A.; Schowalter, W. R. Colloidal dispersions. Cambridge Univ. Press: Cambridge, 1989). These particles can be used as interesting probes for applications such as microrheology (Mason, T. G.; Ganesan, K.; van Zanten, J. H.; Wirtz, D.; Kuo, S. C. Particle tracking microrheology of complex fluids. Phys. Rev. Lett. 1997, 79, 3282-3285; Cheng, Z.; Mason, T. G. Rotational diffusion microrheology. Phys. Rev. Lett. 2003, 90, 018304) or bio-microrheology (Weihs, D.; Mason, T. G.; Teitell, M. A. Bio-microrheology: A frontier in microrheology. Biophys. J. 2006, 91, 4296-4305). Concentrated dispersions of solid shape-designed particles could exhibit interesting liquid-crystalline phases and exotic phase transitions as the particle volume fraction is increased quasi-statically. Moreover, by rapidly concentrating the particles in the liquid, one may quench in glassy disorder (Torquato, S.; Truskett, T. M.; Debenedetti, P. G. Is random close packing of spheres well defined? Phys. Rev. Lett. 2000, 84, 2064-2067). Understanding how the shape of the particles can influence jamming (Donev, A.; Cisse, I.; Sachs, D.; Variano, E. A.; Stillinger, F. H.; Connelly, R.; Torquato, S.; Chaikin, P. M. Improving the density of jammed disordered packings using ellipsoids. Science 2003, 303, 990-993) in concentrated dispersions can provide key insights into the structure and dynamics of disordered soft materials.

### P-DePT Examples

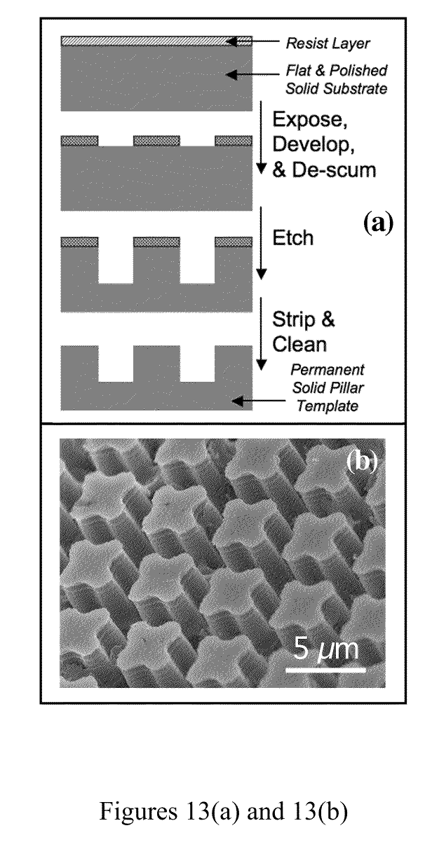

To create a solid pillar-template suitable for P-DePT, as an example, we begin by creating a reticle mask containing a plurality of disconnected cross-shapes suitable for optical lithography; this reticle mask can be designed using computer aided design software and stored electronically in a digital file, and the mask can be produced from the digital file using a standard e-beam lithography writing system (e.g. MEBES). Using a mercury i-line stepper exposure system (Ultratech XLS-7500), ultraviolet light passes through the reticle's clear cross shapes to expose a one micron thick resist-coated (Shipley AZ-5214) flat silicon wafer. Following development and descumming, which removes the unexposed resist from the wafer's surface, a pattern of raised crosses of cross-linked resist remains on the wafer's surface, and the wafer is permanently etched using a reactive ion etcher to a depth of 8 microns in the regions outside the crosses where the wafer is exposed and unprotected. The residual protective resist is then stripped and the wafer is cleaned using piranha (a mixture of 70% sulfuric acid and 30% hydrogen peroxide). This process is shown schematically in FIG. 13(a). The resulting pillar-deposition particle template on a five-inch silicon wafer contains roughly one billion raised pillars that define the desired particle shapes, shown in the scanning electron micrograph of FIG. 13(b). Because the top surfaces of the pillars have been protected by the photoresist, they remain flat. The side surfaces of the pillars may have irregularities; as shown in this example, these will not affect the particle production process by the pillar method. Furthermore, the etch depth has been made larger than the desired thickness of the particles. Extremely high etch depths may not be desirable since pillars would become more susceptible to breakage from accidental mechanical contact or agitation of the template. Generally, an etch depth of at least twice the maximum particle thickness is appropriate. Other alternative approaches that yield the same permanent pillar template structure, such as depositing a silicon oxide layer onto a silicon wafer, performing resist-based lithography to print the repeating disconnected patterns of particle shapes, and etching the silicon dioxide, could also be used.

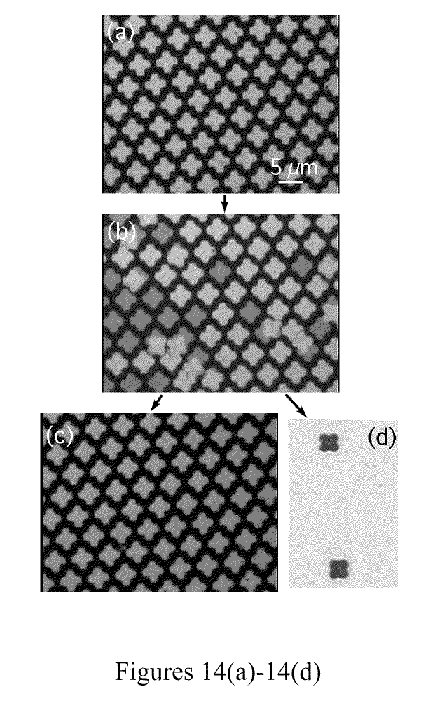

Using the pillar-template, as an example, we produce an aqueous suspension of cross-shaped gold plate-like particles according to general scheme of FIG. 12. Since surfaces containing pillars, such as lotus leaves, are known to produce high effective contact angles for liquids that can make deposition of liquid-based polymer solutions problematic, an adhesion promotor, HMDS, is applied to the silicon by vapor condensation. Next, a thin water-soluble sacrificial release layer (e.g. Omnicoat) is then spin-coated at three thousand RPM to provide a thickness of approximately 20 nm onto the pillars and then baked at 200 ° C. for 1 minute. The desired thickness of gold, 45 nm, is deposited uniformly onto the surface using sputtering. An optical micrograph of the top surface of the coated pillars is shown in FIG. 14(a). Following the deposition of the desired particle material, the coated pillar-template is immersed in water, and agitated to cause the sacrificial layer to dissolve and the particles to be released into solution, as shown in FIGS. 14(b)-14(d). Typically, 2 minutes of agitation in an ultrasonic bath is adequate. Care must be taken so that the intensity of ultrasonic agitation is not so severe that it would cause released particles to break apart or damage the pillars on the template.

Using scanning electron microscopy, we have characterized the number-weighted polydispersity of the arm lengths and widths of the crosses to be about 2%. The polydispersity of the thickness is more difficult to measure for such a thin layer, and we estimate it to be about 45±5 nm. The polydispersity of the edge lengths is essentially set by the precision of the pillar template (i.e. through the exposure and etching processes), and the polydispersity of the thickness by the uniformity of the deposition process for coating the pillars with the desired materials. In general, for directional deposition of the desired particle material, we do not observe overhangs, burs, or other defects, and the side-walls are flat. Other forms of deposition, such as solution delivery of a desired organic material, to the tops of the release-coated pillars and subsequent baking could lead to rounding of the top corners of the particles by liquid surface tension. This may be a desirable feature in some cases.

The P-DePT process can be repeated many times without degradation of the pillar template. If deposited materials accumulate in the interconnected trenches beneath the pillars, occasionally, it may be necessary to clean off this excess material by dipping the wafer in piranha or HF solutions. If the trenches are also coated with a release agent when the tops of the pillars are coated, then large continuous interconnected regions of the deposited material containing negative images of the desired particles can also be released into solution. These regions can be easily separated from the particles through sedimentation or filtration, since they are typically tens to hundreds of microns in size.

We have characterized the rate of efficiency of the lift-off of the particles from the pillars by using optical reflection microscopy to examine the tops of the posts after the sacrificial layer has been dissolved. When the sacrificial layer is properly coated over all of the tops of the pillars, it is very difficult to find any gold crosses that remain on the posts after the fluid assisted release step, and we estimate the efficiency of release of the particles to be greater than 99%, with less than one particle in ten thousand remaining on the wafer. The few bound particles that do remain are found near the edges of the wafer where the spin-coating of the release agent may have been adversely affected by the high effective contact angle introduced by the pillars. Vapor deposition of the sacrificial layer, rather than spin-coating, would most likely improve the release efficiency. The simplicity of release and the exceptional release efficiency is one of the strengths of the P-DePT approach.

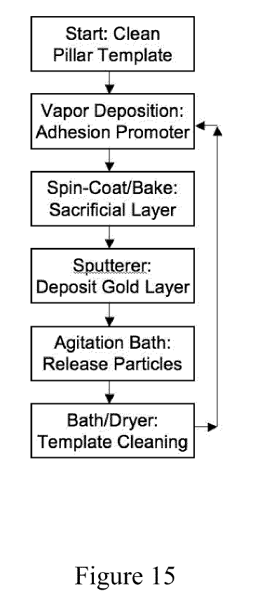

To continuously produce particles at a high rate, an automated system containing the essential non-optical devices for each step in the above process can be set up in a continuous loop. For the example we gave, several identical pillar templates held in a wafer boat can be fed by an automated robotic track system into a hexamethyldisilizane (HMDS) applicator, a spin-coater, a baker, a sputterer, an ultrasonic bath, a cleaning tank, a drying stage, and then back to the HMDS applicator to complete the loop (FIG. 15). The HMDS applicator, spin coater, baker, cleaning tank and/or a drying system can be components of a template cleaning and preparation system of a system for manufacturing particles according to an embodiment of the current invention. The sputterer is one example of a possible deposition system for manufacturing particles according to an embodiment of the current invention. The deposition system is not limited to only a sputterer and may include other deposition systems including those described in references to various examples herein. The ultrasonic bath is one example of a possible particle removal system according to an embodiment of the current invention. However, systems for manufacturing particles according to various embodiments of the current invention are not limited to this specific example. The track system is only one possible way of performing high-throughput production. A rotating carrousel of identical pillar templates could also be used. Alternatively, various operations can be performed on only a region of the wafer as it is rotated or translated, if appropriate deposition methods are used. With such an automated system, we estimate that roughly 10¹¹ microscale particles can be made per day per wafer without the need for human intervention. For submicron particles, the rate of production could far exceed 10¹¹ particles per day per wafer. By producing particles from multiple templates simultaneously, the production rate can exceed that of particle production methods relying on spatially patterned radiation.

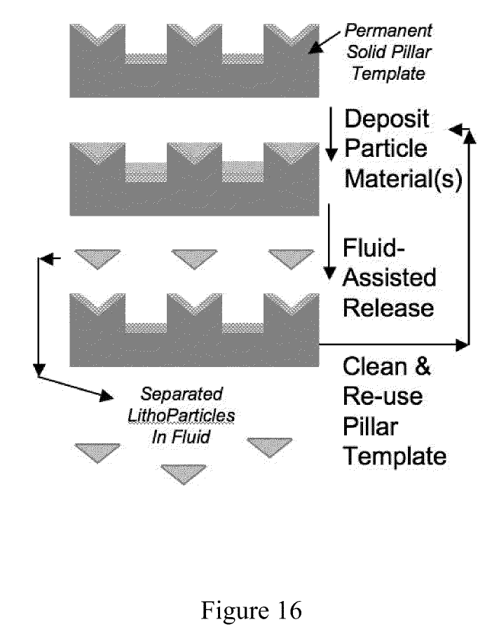

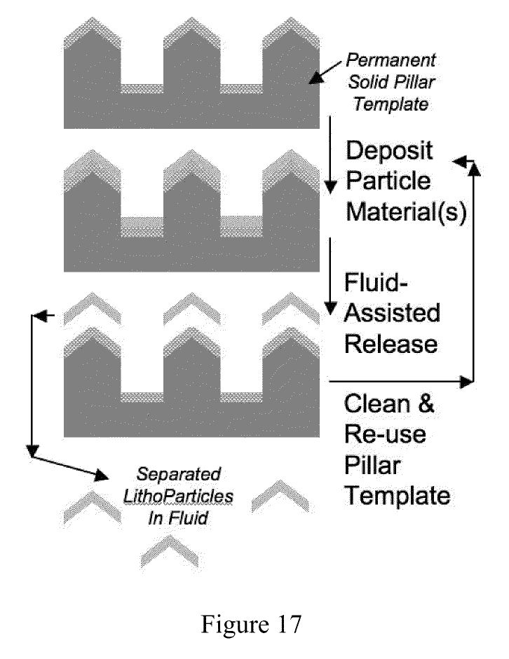

Although P-DePT is well suited for making particles that are slab-like and have a uniform thickness, it is also possible to make particles that have more complex three-dimensional shapes by appropriately modifying the surfaces of the pillars. For instance, it is possible to make a pillar-template suitable for creating pyramid-shaped particles by filling the trenches of the well-template with an inert material, leaving the tops of the pillars exposed, and then etching the tops of the pillars at an angle, as can be achieved by angular etching of an appropriately oriented polished silicon wafer surface. After etching, the surfaces of the pillars can be coated with a release agent. As shown in FIG. 16, deposition of the desired particle material onto the pillars and subsequent release by fluid agitation yields an efficient non-optical process for producing complex LithoParticles. By depositing a uniform layer of the desired particle material onto pillars that are not flat, one can create non-planar particles that have uniform thickness, yet retain the contours of the tops of the pillars, as shown in FIG. 17.

In addition to gold particles, we have produced plate-like square-cross particles made of aluminum that have thicknesses in excess of one micron, showing that P-DePT can be used to fabricate particle structures that are quite thick and robust. The ultimate limit of the particle thickness is set by the height of the pillars; if the wells outside of the pillars become filled with the particle material, then the particle material will form a continuous interconnected layer, and no particles can be produced. However, if the height of the permanent pillars is larger than the lateral dimensions of the particles, as it is in our example, then the thickness of the particles can actually exceed the lateral dimensions, without a loss in the definition of the lateral shape. So, both thin and thick particles can be made using the P-DePT method.

Residual stress in the layer of deposited particle material can cause the particles to deform into non-planar shapes, especially when the thickness of the deposited layer is much less than a micron. This effect has been reported previously (Brown, A. B. D.; Smith, C. G.; Rennie, A. R. Fabricating colloidal particles with photolithography and their interactions at an air-water interface. Phys. Rev. E 2000, 62, 951-960), but, in our method, the gold particles remain quite planar, even after release, as can be seen in the optical micrographs. Further electron microscopy shows that the gold particles do not exhibit significant distortions away from planar shapes. In principle, by depositing a thin layer of a particle material that is known to have an inherent stress, it could be possible to design continuously curved particle shapes. Indeed, by relying upon stresses created by controlling the composition (e.g. stoichiometry) of multi-elemental particle materials, one can induce a desired curvature after lift-off. One can also create bilayer deposition of two desired particle materials that have different thermal coefficients of expansion, yielding two-faced Janus particles that have continuously variable shapes that can be controlled as a function of temperature. This can be accomplished by simply depositing a layer of one desired material, and then a second layer of a different desired material having a different coefficient of thermal expansion onto the tops of the pillars before releasing these bi-layer particles into a fluid.

Many different deposition scenarios, both for the sacrificial layer and for the target material layer, are possible once the permanent pillar template has been made. These materials include organics (e.g polymers), biomaterials, inorganics (e.g. nitrides and oxides), and metals, or combinations of any of these compositions. Performing multiple depositions of different desirable target materials prior to the release step can be used to make hybrid multi-layer particles. These deposition methods include, but are not limited to, spin-coating, spray-coating, dip-coating, sputtering, physical vapor deposition (PVD), chemical vapor deposition (CVD), molecular beam epitaxy (MBE), and electron-beam metal deposition (EBMD). Directional deposition at other than normal to the pillar's top surface could provide a method of making particles with slanted side-walls. Release can be made into aqueous or non-aqueous solvents for further chemical surface treatment to increase particle stability against aggregation. Particle release could take place in any fluid, including supercritical fluids or gases, not just liquids. Lastly, it may be possible to omit the sacrificial layer if a suitable surface coating can be used to prevent the particles from sticking to the pillars. Such a permanent coating may take the form of fluorinated molecules that are attached in high density to the template surfaces.

In P-DePT, we employ a re-usable patterned substrate with permanent pillars and do not require exposure by any source of radiation, thereby clearly differentiating this approach from earlier optical approaches of Higurashi et al.( Higurashi, E.; Ukita, H.; Tanaka, H.; Ohguchi, O. Optically induced rotation of anisotropic micro-objects fabricated by surface micromachining. Appl. Phys. Lett. 1994, 64, 2209-2210), Brown, et al. (Brown, A. B. D.; Smith, C. G.; Rennie, A. R. Fabricating colloidal particles with photolithography and their interactions at an air-water interface. Phys. Rev. E 2000, 62, 951-960), Harrison, Chaikin, and Mason (Sullivan, M.; Zhao, K.; Harrison, C.; Austin, R. H.; Megens, M.; Hollingsworth, A.; Russel, W. B.; Cheng, Z.; Mason, T. G.; Chaikin, P. M. Control of colloids with gravity, temperature gradients, and electric fields. J. Phys. Condens. Matter 2003, 15, S11-S18), and Hernandez and Mason (Hernandez, C. J.; Mason, T. G. Colloidal alphabet soup: Monodisperse dispersions of shape-designed lithoparticles. J. Phys. Chem. C 2007,111, 4477-4480). P-DePT can offer a clear advantage of a re-usable permanently patterned template, excellent uniformity, and high-throughput without the complexity of optical exposure at every stage in the process. Because a stamping, or “imprinting” procedure (Chou, S. Y. Nanoimprint lithography and lithographically induced self assembly. MRS Bulletin 2001, 26, 512; Chou, S. Y.; Krauss, P. R.; Renstrom, P. J. Nanoimprint lithography. J. Vacuum Sci. Tech. B 1996, 14 (6), 4129-4133; Resnick, D. J.; Mancini, D.; Dauksher, W. J.; Nordquist, K.; Bailey, T. C.; Johnson, S.; Sreenivasan, S. V.; Ekerdt, J. G.; Willson, C. G. Improved step and flash imprint lithography templates for nanofabrication. Microelectronic Engineering 2003, 69, 412-419), in which particles can potentially be stuck in wells with vertical side-walls that can inhibit facile release, is not necessary, we anticipate that P-DePT will be more efficient than other particle methods involving mechanical imprinting that we have also developed. Moreover, no special fluorinated surface coatings or expensive mechanical imprinting stages are required. The internal feature sizes and overall dimensions of the particles are not limited to the microscale; direct e-beam writing to a resist-coated surface or deep-UV lithography and subsequent etching could make templates with internal particle features, such as arm widths on the crosses, and overall particle lateral dimensions, smaller than 50 nm.

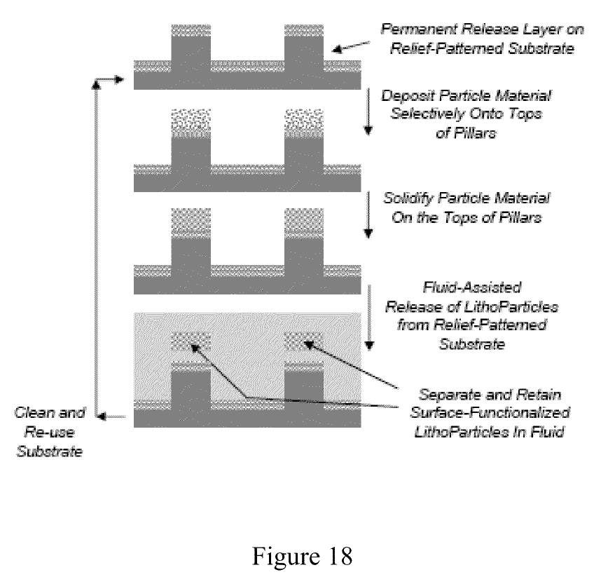

FIG. 18 is a schematic illustration of another embodiment of P-DePT according to the current invention. This example takes advantage of the wetting of only the tops of the pillars that is common when a liquid material is coated onto a pillar template. If the pillars on the pillar template are spaced close enough together, many liquids will be confined to the top surfaces of the pillars and will not penetrate into the troughs below. The deposition of the liquid can occur through spray-coating, spin-coating, dip-coating, painting, or other methods. Solidification can occur by thermal processes, chemical processes such as crosslinking, or through evaporation of a carrier solvent that may contain dispersed materials. Some advantages of this method can include: (1) the particle material is deposited only in the regions that will lead to the desired particles, so the particle material is more efficiently used, and (2) cleaning the substrate is easier at a later stage in the process.

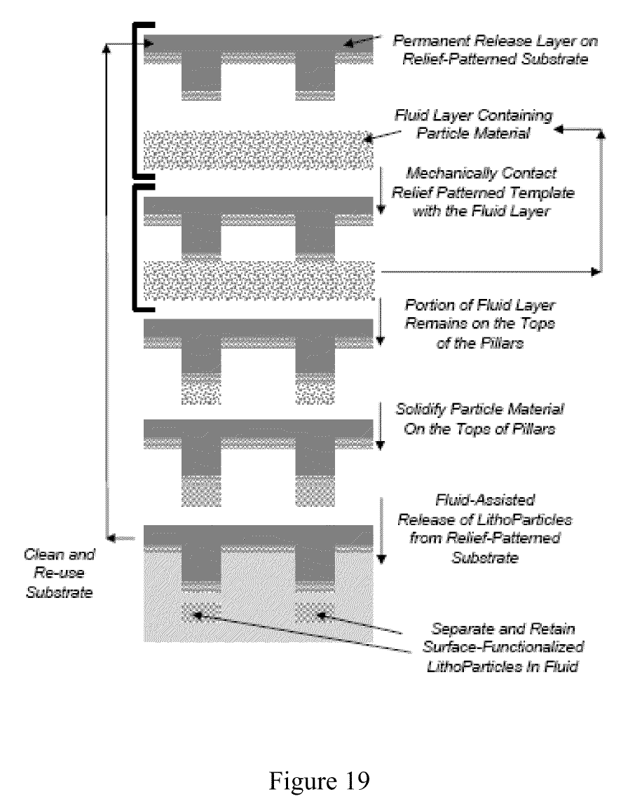

FIG. 19 is a schematic illustration of another embodiment of P-DePT according to the current invention. This example takes advantage of the wetting of only the tops of the pillars that is common when a liquid material is coated onto a pillar template. If the pillars on the pillar template are spaced close enough together, many liquids will be confined to the top surfaces of the pillars and will not penetrate into the troughs below. The deposition of the liquid can occur through spray-coating, spin-coating, dip-coating, painting, or other methods. Depositing viscoelastic materials such as concentrated polymer solutions or polymer melts, on the tops of the pillars can be advantageous since the elasticity inherent in the viscoelastic material can inhibit the formation of undesirable bridges of the material between adjacent pillars. Eliminating liquid bridges that may occur between the top surfaces of adjacent pillars can be achieved by spinning the template at a higher speed or by applying an external fluid flow, acoustic field, mechanical vibration, or electric field. Solidification can occur by thermal processes, chemical processes such as crosslinking, or through evaporation of a carrier solvent that may contain dispersed materials. Some advantages of this method can include: (1) the particle material is deposited only in the regions that will lead to the desired particles, so the particle material is more efficiently used, and (2) cleaning the substrate is easier at a later stage in the process.

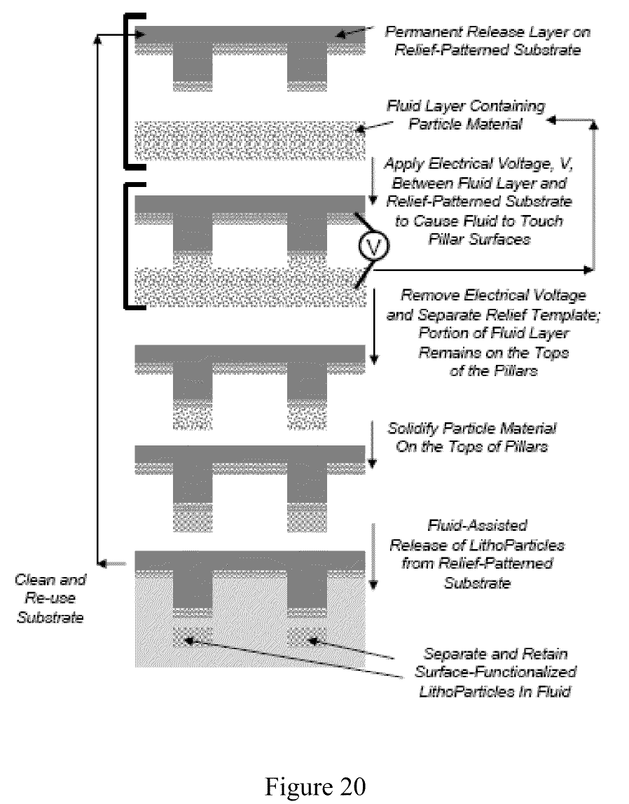

FIG. 20 is a schematic illustration of another embodiment of P-DePT according to the current invention. Applying an electric field through a voltage (i.e. potential difference) between the fluid layer and the relief template can cause the particle material to wet the extreme surfaces of the pillars. Solidification can occur by thermal processes, chemical processes such as crosslinking, or through evaporation of a carrier solvent that may contain dispersed materials. Some advantages of this method can include: (1) the particle material is deposited only in the regions that will lead to the desired particles, so the particle material is more efficiently used, and (2) cleaning the substrate is easier at a later stage in the process. This process can be repeated to produce bi-layer or multi-layer LithoParticles.

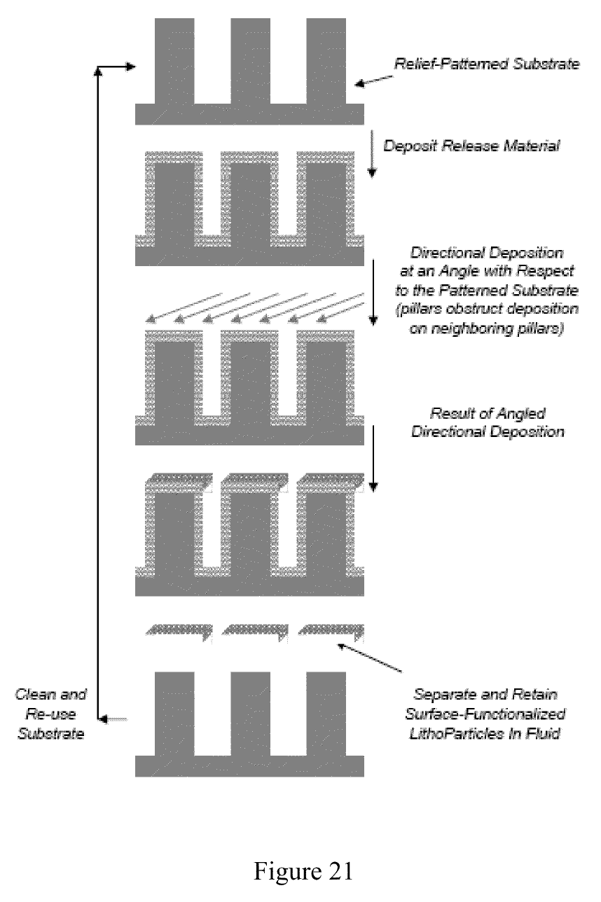

FIG. 21 is a schematic illustration of another embodiment of P-DePT according to the current invention. This example uses angled directional deposition to create LithoParticles that have non-slab shapes. Although directional deposition is usually along a direction parallel to the pillar axes (i.e. straight down from the top of the page), angled deposition, in which the direction of the motion of the deposited material is not aligned along the pillar axes, can also be used to create more complex shapes. The same kind of angled deposition could be made using well-templates, not only pillar templates.

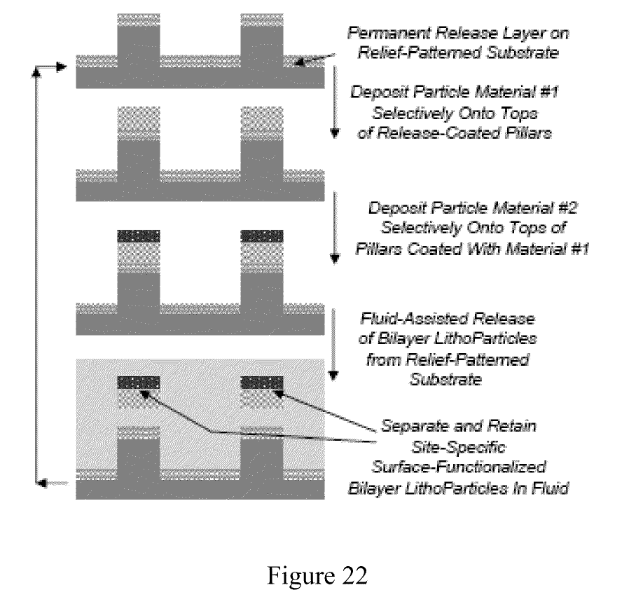

FIG. 22 is a schematic illustration of another embodiment of P-DePT according to the current invention. This example takes advantage of the wetting of only the tops of the pillars that is common when a liquid material is coated onto a pillar template. In this particular example, only two different particle materials have been added to the tops of the pillars in sequence to create bi-layer lithoparticles. This procedure can be extended to add additional layers of the same or different particle materials to build up multi-layer lithoparticles. Liquid deposition to the tops of the pillars is just one way to create the particles; other forms of deposition in a desired sequence could be used to create and customize additional layers of different types of materials that form the particle material.

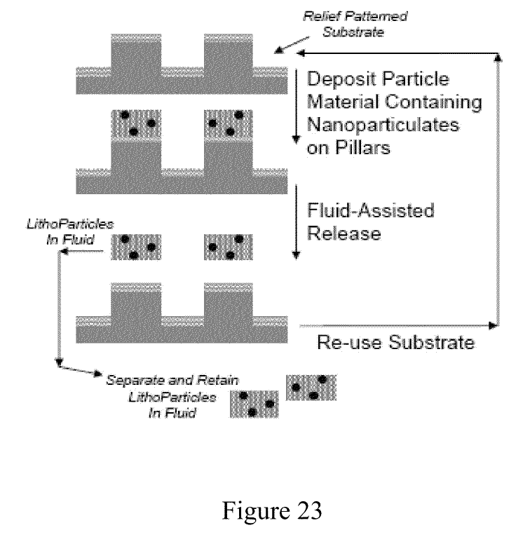

FIG. 23 is a schematic illustration of another embodiment of P-DePT according to the current invention. In this example, microscale particles (e.g. polystyrene spheres, silica spheres, clay), nanoscale particles (e.g. iron oxide, quantum dots, dendrimers), and molecular species (e.g. star polymers, plasticizers, proteins, polypeptides, dyes) can be incorporated into the matrix of the particle material to form a customized complex composition.

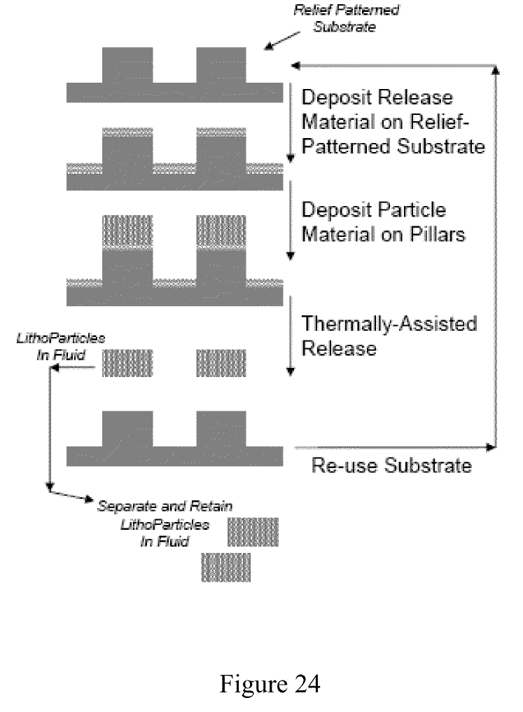

FIG. 24 is a schematic illustration of another embodiment of P-DePT according to the current invention. Rather than using fluid-assisted release (with or without agitation), as in some of the other examples described, the LithoParticles are released from the substrate by changing the temperature. As an example, the release material could consist of a solid over a range of temperatures used to form particles; subsequently, the temperature is changed out of this range to cause the release layer to become fluid, thereby liberating the lithoparticles from the substrate. Optionally, this approach could be done with or without the presence of a fluid into which the lithoparticles would be dispersed.

### Further Embodiments

Both pillars and wells can be made on the same template surface to yield a mixed template that can produce particles by both processes. For this kind of mixed template, the same deposition step can create discrete disconnected regions in the form of the desired particles on the tops of the pillars and in the bottoms of the wells simultaneously. A single lift-off step can release the particles both from the pillars and from the wells.

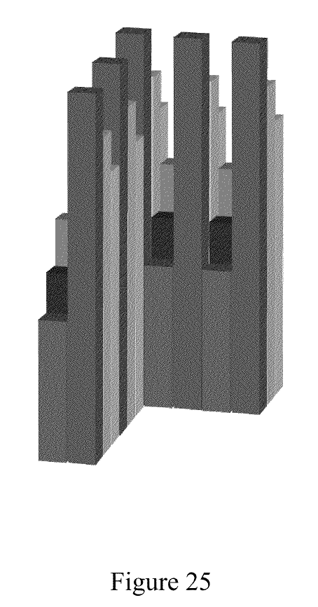

More generally, the solid template can be created in such a manner as to provide several different plateau levels at different depths from its topmost surface upon which the desired material can be deposited. The desired material can be deposited in a manner that leaves disconnected regions of this material at different levels in the form of the desired particle shapes. These disconnected regions can be released from the template, yielding particles in solution. In principle, using this approach, all of the deposited material can be used to form desired particles without waste, provided the different shapes can be formed on the template at different levels and completely fill the available surface area. This would be a highly efficient implementation that would make excellent use of the deposited material. The example in FIG. 25 shows a template for making square shaped particles comprised of six different levels that are arranged in a pattern so that no two levels of the same height are neighbors when repeated everywhere over the surface of the template. Each of the levels could have additional surface features that can be used to create texturing, asperities, bonding sites, or indentations on the surfaces of the particles that are produced. Directional deposition of the desired particle material from above onto this template will result in identical square particles that are disconnected from each other being produced over the entire surface of the template. A single release step can release the particles from all of the different levels simultaneously, and the template can be re-used. In general, the profile of the top surface can be of a shape other than a square (e.g. square crosses, Penrose tiles, etc.) could be used. Several different shapes can be tiled at different levels onto the same template. The simple example for squares in FIG. 25 illustrates the more general type of template that can be used to make particles by a template deposition process.

Templates can potentially have many different forms other than being made on a flat wafer surface. The overall template surface does not have to be flat for either the pillar deposition templating process or the well deposition templating process in order to produce useful particles. For instance, a template can be made on a curved surface, such as a cylinder, which could be spun to expose different portions of the cylinder to cleaning, deposition, and release processes. Using such a curved template that has appropriate pillars and/or wells on the surface, one may be able to optimize the processing steps into a continuous particle production device that does not require repeated exposure with radiation. Templates made from flexible solid materials could be adhered to a solid surface. Well templates could potentially be made by making a thin porous film of a flexible solid material that has holes of the desired particle shape and then adhering this film to a non-porous solid support. Indeed, lifting off the top contiguous layer of the simple well deposition templating process could potentially produce a film that could be used, in turn, to make another well template if this film is deposited and bonded to a solid support.

Templates can be made by many different possible procedures. Standard lithography procedures, such as electron beam lithography and optical lithography, can be used in conjunction with etching, to make the templates. However, other methods can be used, too. One method involves coating a wafer surface with diblock polymers that form phases of dots or short stripes that can be etched onto the wafer's surface to provide either pillars or wells in the form of the dots or stripes. Another possible method is to coat the wafer surface with a solution of polymer particles and use these particles as a mask during an etching process. This type of process could be used to make circular pillars or even ring-like pillars. If complex particle shapes, such as those made using lithographic methods, are deposited, templates for reproducing their shapes could potentially be made this way. Yet another method of making a template could be to cover a wafer's surface with a microporous or nanoporous membrane or film. This kind of well template may not be comprised of only one material but may be made instead from two or more materials that have been put together to create the desired pillars and wells. Optionally, the exposed surface of the wafer could be selectively etched using an ion etcher in the regions where the holes appear and the membrane could then be removed from the surface.

Multiple deposition steps using different materials can be used in combination with templates in order to make complex particles that have layers of different kinds of materials, including organics, inorganics, metals, alloys, and biomaterials. By combining sequences of deposition of different desired materials in controlled amounts with complex templates that have multiple levels in different shapes, it is possible to produce very complex particles that have differently shaped substructures of particularly desired materials located in pre-specified regions. In particular, selective spatially patterned deposition can be used in combination with the templates to create local sites for producing pre-specified interactions, whether attractive or repulsive, between different particles. Alternatively, local regions on the surfaces of the particles can be made rough through a selective deposition process that coats only part of the particles' surfaces with a desired material in a manner that produces an enhanced surface roughness in a desired sub-region of the particle. Thus, by controlling the deposition as well as the template, it is possible to design particles that have customized localized surface coatings that can interact with local sites on the surfaces of other particles to form assemblies of particles that have either the same or different shapes.

Before the particle is separated, it typically will be or will become at least partially solid so that it retains a geometrical feature of the surface portion of the template (or coated template) that it was in contact with, after the separation. The forming of a particle could involve depositing a liquid dispersion and then inducing a chemical reaction, thermal polymerization of a polymer component, photo-induced polymerization, plasma-induced polymerization, sintering, a crosslinking reaction, a gelation, an evaporation of the solvent, an aggregation or agglomeration of materials, a jamming, an entanglement, a denaturation, and/or a bonding.

The constituent material as first applied to the template can be a vapor, a liquid, or a solution, for example. The maximum dimension associated with any of the components contained within the constituent material should be smaller than the maximum dimension associated with the portion of the surface for creating the particles. For example, it may not be reasonable to coat the surfaces of the pillars with giant particles that are larger than the pillars themselves.

The structured substrate can be produced from a flat smooth substrate by a lithographic process involving at least one of electron-beam lithography, optical lithography, ultraviolet lithography, dip-pen lithography, x-ray lithography, imprinting, stamping, deposition, patterning, and etching.

### Surface Modifications of Particles

Some strategies for directing the assembly of colloidal particles can involve site-specific interactions between a portion of the surface of one particle and the portion of a surface of a second particle. Some surface-surface interactions can be repulsive, some can be attractive, and there can be variations of these interactions depending upon the nature of different stabilizing materials attached to the surfaces of the particles. The processes of attachment, usually through means such as deposition, adsorption, or bonding, can be material-specific and shape-specific. Therefore, it is important to develop method for producing particles that have custom-modified surfaces that can interact in pre-specified and desirable ways. The surface modifications can be designed to create multi-component assemblies of the particles that are based on controlling the attractive and repulsive interactions between different portions of the surfaces of the particles through selective surface treatments that can be localized.

Further embodiments of the invention include methods to modify the surfaces of particles that are produced using relief deposition templating (RDT). The fundamentals of the process for making particles using relief deposition templating (i.e. through processes such as pillar deposition templating (P-DePT) or well deposition templating (W-DePT)) is described above and in references. (Hernandez et al., “Pillar-Deposition Particle Templating: A High-Throughput Synthetic Route for Producing LithoParticles, ” Soft Materials, vol. 5, pp. 1-11, 2007; and Hernandez et al., “Well-Deposition Particle Templating: Rapid Mass-Production of LithoParticles Without Mechanical Imprinting,” Soft Materials, vol. 5, pp. 13-31, 2007)

Variations of the process of making particles using RDT couple the steps of the process of forming the particles described previously with the steps of modifying their surfaces. The specific steps to decorate specific portions of the particles are sufficiently complicated and involved so as to be non-trivial and non-obvious. These further steps may be used with RDT, but may also be applicable to methods of making lithographic particles using spatially patterned radiation (Hernandez et al., “Colloidal Alphabet Soup: Monodisperse Dispersions of Shape-Designed LithoParticles,” J. Phys. Chem. C, vol. 111, pp. 4477-4480, 2007) and also to relief radiation templating (US Provisional Application 61/103677, incorporated by reference in its entirety).

Coating of the entire surfaces of particles after release from the substrate is typical for the purpose of stabilizing the particles against agglomeration by attractive interactions and thermally driven aggregation. However, this simple process of coating cannot create the complex decorations over portions of the surfaces of particles that may be necessary to design and build multi-component colloidal structures.

One method of creating patches (i.e. portions of the surface of a lithographic particle) of different materials on the surfaces of particles is based on lithographic patterning (e.g. through deposition, exposure of a radiation-sensitive resist, development, and etching). In some cases, no further surface treatment is necessary and the patches created through patterning are all that are necessary to achieve the desired surface modification. In other cases, the patches of deposited material over a portion of a particle's surface serve as localized regions that facilitate the selective attachment of molecules in solution or other colloidal species (i.e. objects) dispersed in solution only onto the patches and not onto the other surfaces.

As an example, we consider the modification of patches by molecules that can be designed to interact with different strength and range. Such molecules typically have a region that enables them to attach to the particle material and a region that enables them to interact with the surfaces of other particles or with molecules or other species that are on the surfaces of other particles. Such multi-functional molecules are sometimes referred to as “functionalized” or “derivatized” molecules (Malmsten et al, “Biopolymers at Interfaces,” Vol. 110 of the Surfactant Science Series, 2nd. ed., Taylor and Francis, 2003). In some cases, certain molecules (e.g. derivatized single-stranded oligomeric DNA) can be designed to have a functionality that enables them to bind to complementary molecules (e.g. derivatized single-stranded oligomeric DNA that has a complementary sequence of nucleic acids) that can likewise be attached to patches on other particles. If the attractive binding of one or more molecules (e.g. that have been previously attached to the surfaces of particles) occurs, and if the attractive binding energy exceeds thermal energy, then the particles having patches containing complementary particles can be strongly bound together without coming apart due to thermal fluctuations as they remain in the suspending fluid material. For example, a first particle having a patch of material onto which derivatized oligomeric single-stranded DNA molecules have been attached can be bound to a second particle having a patch of material onto which different and complementary derivatized oligomeric single-stranded DNA molecules (ss-DNA oligos) have been attached. The binding can occur when, through diffusion, flow, or manipulation by external fields, the first particle can be brought into the vicinity of the second particle with an appropriate position and orientation for the binding to occur. In some cases, it may be advantageous to extend the ss-DNA oligos beyond the surfaces of the particles by placing them on a polymer strand (i.e. like a stalk) that holds the oligos away from the particle surface, thereby letting complementary oligos bind more readily as entropic forces enable them to explore more space as they dangle and undergo thermal fluctuations. This polymer strand might be a segment of double-stranded DNA that would not interfere with the interaction of the ss-DNA.

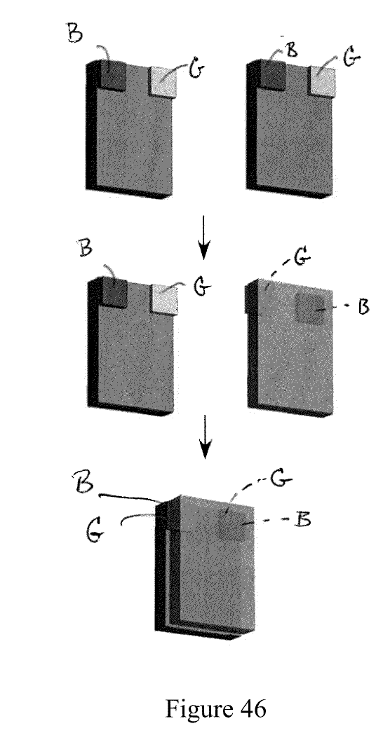

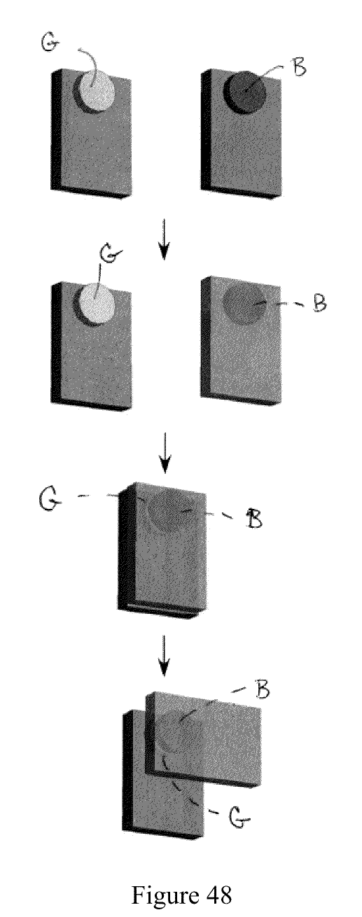

In some embodiments, binding of molecules on a first patch on a first particle to complementary molecules on a second patch on a second particle may lead to several different degrees of relative motion of the particles. For instance, binding of two particles that arises from a single pair of complementary molecules on proximate patches may lead to a bond that prevents the particles from coming apart under thermal excitations, flow, or external fields; however, the two particles may be able to rotate with respect to each other if the bond has little resistance to twisting (illustrated in FIG. 48). This can be a desirable consequence. One way of obtaining this may be to reduce the surface density of the molecules on the patch to be very low, so that only a few molecules are present on a particular patch. However, in other embodiments, it can be desirable to create a shear-rigid bond between the particles. This can be accomplished by binding two or more pairs of complementary molecules on the surfaces of the same proximate patches (illustrated in FIGS. 46, 47 and 49). The additional bonds provide resistance to torques that might cause modes of relative rotation.

The strength of the binding between different types of patches can be selectively controlled through the temperature and the ionic strength of the suspending fluid material. For instance, the strength of bonds between complementary ss-DNA oligos can be greatly reduced by heating the suspension of particles to temperatures that are typically below the boiling point of the suspending material (e.g. up to about 100° C. for aqueous nucleic acid materials). The larger thermal energy effectively overcomes the attractive energy between the complementary molecules, and the molecules dissociate. By controlling the length and complementarity of the sequences of the ss-DNA oligos on the proximate patches, it is possible to adjust the dissociation temperature of molecules on different patches on the same particle surfaces. Thus, by heating the suspension of particles that contain patches of the complementary particles, it is possible to create a stable suspension of individual unbound particles. Then, by lowering the temperature below a first dissociation temperature associated with a first pair of complementary ss-DNA oligos, it is possible to cause only a specific first patch on one particle to associate with a specific second patch. Usually, a higher dissociation temperature is associated with longer oligomeric sequences that have a significant degree of complementarity. Waiting sufficient time enables a first binding of the particles to occur (e.g. as thermal energy drives the particles close enough to each other to enable the complementary molecules on patches of particles to come into proximity so that binding can readily occur). Subsequently, the temperature is then lowered below a second dissociation temperature associated with a second pair of complementary ss-DNA oligos. Waiting a sufficient time enables a second binding of particles to other particles and/or to multi-particle structures that have been previously formed through the first binding to occur. Repeating this process of changing temperature and waiting for that step of the assembly to occur can thereby enable mass production of multi-component assemblies of one or more types of particles by designing the location and appropriate molecular types of a plurality of patches on the surfaces of said one or more types of particles. This step-wise method of causing particles to assemble could even be potentially used to create a self-replicating colloidal system based on templates that are grown through a step-wise hierarchical assembly process.

As another example of this approach, the temperature could be fixed, but the ionic strength of the fluid material in which the surface-treated particles are suspended may be likewise systematically controlled. Certain patches on the surfaces of particles may be modified with surface treatments to respond to changes in the ionic strength of the fluid material (e.g. by adding salts or saline solutions to the fluid material in which the particles are suspended). Similar extensions of this approach for changes in pH (e.g. by adding acids or bases) or other physiochemical variables are likewise anticipated.

The use of intermediate linker molecules of ss-DNA that contain sequences of nucleic acids may also be introduced after the surface treatment steps to induce attractive linking. These linker molecules would contain at least complementary portions of the sequences that are on the ss-DNA attached to the surfaces of the particles that have been designed to be bound together. It can be anticipated that intermediate linkers other than ss-DNA that interact with the molecules or other species on the modified surfaces of particles could also be added to bind together specific sites on the surfaces of two or more particles to initiate the creation of multi-component assemblies.

Note that herein, although we use the term oligomeric to refer to bound polymer-like molecules, we intend to mean by this term both shorter oligomeric molecules and also longer polymeric molecules and do not intend to imply any strict restriction on the length of the molecules.

Although the examples of the embodiments of this invention are primarily focused on the design of the molecular or other species bound to specific portions of the surfaces of the particles, it is this design that enables the broader concept outlined of creating multi-component assemblies through control over the surface interactions between one or more particle species that can be caused to bond together in an predetermined and desirable manner. This approach enables the creation of colloidal devices and machines out of lithographic particles in a highly parallel manner that is off-chip and is typically in the suspending fluid material.

An example of a specific type of linkage that can be used to attach derivatized molecules is the thiol linkage resulting when thiolated derivatized molecules are attached to a patch composed of gold. Other types of materials-specific surface chemistries can be used to attach molecules and particles, as are commonly known in the art through references on surface chemistry and molecular derivatization (Malmsten et al, “Biopolymers at Interfaces,” Vol. 110 of the Surfactant Science Series, 2nd. ed., Taylor and Francis, 2003). Complementary natural and synthetic biopolymers that have amino acid sequences that create attractive interactions between the biopolymers (e.g. the streptavidin-biotin pair) can also be used instead of complementary ss-DNA oligos as the linking molecules.

Although we refer to molecules bound to patches, other species can also be attached to patches in order to provide site-specific binding of a patch on a particle with a patch on another particle. These other species include: nanoparticles, molecularly-coated nanoparticles, dendrimers, microgels, nanoemulsions, double nanoemulsions, vesicles, viruses, viral capsid proteins, peptides, oligopeptides, polypeptides, block copolypeptides, graft copolypeptides, block copolymers, proteins, biopolymers, organelles, membranes, lipids, lipoproteins, amphiphilic molecules, derivatized molecules, molecular motors, derivatized molecular motors, structural biomolecules, derivatized structural biomolecules, membrane biomolecules, and derivatized membrane biomolecules.

Attachment of molecularly coated nanoparticulate materials onto a patch can be used to control surface roughness of that patch in addition to the molecular surface chemistry on that patch. Since surface roughness has been shown to strongly influence depletion attractions (Zhao et al., “Roughness-Controlled Depletion Attractions for Directing Colloidal Self-Assembly,” Phys. Rev. Lett., vol. 99. pp. 268301/1-4, 2007), then the attachment of coated nanoparticulate materials on patches on micron and sub-micron particles can provide a particularly versatile method of performing assembly that can be controlled by a combination of binding of complementary molecules and of surface roughness.

In some cases, the molecules or other species can be attached by exposing patches created on certain portions of the surfaces of particles to a fluid surface-treatment material that contains a plurality of molecules that readily bind, attach, or adsorb onto the surfaces of the patch. The density of molecules or other species attached to a patch can be controlled by the concentration of molecules in the fluid surface-treatment material, the time the particles are exposed to the fluid surface-treatment material, and the rates of reaction of the molecules or other species with the patch material on the surface of a particle. If some surfaces of the particle cannot be exposed to the fluid surface-treatment material, then such unexposed surfaces will not have the molecules attached to them. Even different portions of the surfaces of particles that do not have any patches and are comprised of only one material can be treated in this manner. For bi-layer or multi-layer particles that are made using different materials for two or more layers, the specific attractive interactions between particular molecules and the layer materials can be used to provide methods of attaching several different molecular types to the various layers, either before or after the release of the particles from the substrate. In some cases, to obtain a high level of complexity, pre-release treatment of particles using a fluid surface-treatment material can be combined with release and post-release treatment of particles, potentially using different fluid surface-treatment materials.

After use of a fluid material containing molecules or other species for pre-release, release, and post-release treatment to attach molecules or other species onto specific portions of the surfaces of the particles, it is frequently necessary to remove the fluid treatment material in order to proceed to the next step. For pre-release treatments, it is possible to simply immerse the substrate to which the particles are bound into a container that holds a sufficient quantity of pre-release fluid surface-treatment material for a predetermined and sufficient length of time for the desired attachment to occur, and to then simply remove the substrate from the pre-release fluid surface-treatment material. Subsequently, the particles and substrate upon which they are bound can be washed by a fluid material that does not cause unintended release of the particles and also does not cause the molecules or other species to detach from the portions of the surfaces of the particles that remain bound to the substrate. In some cases, molecules or other species can be included in the fluid release material in order to cause attachment of a portion of these molecules or other species

Usually, in a single surface-treatment step, the attachment of molecules or other species creates at most a monolayer of those molecules or other species on a portion of the surfaces of the particles. In some cases, it could be desirable to attach molecules or other species to form two or more layers.

A fluid surface-treatment material consists of a fluid material (e.g. a liquid, liquid mixture, supercritical fluid, or a gas) in which molecules or species of a colloidal material are dissolved or dispersed. These molecules or colloidal species have a preference to attach to at least a portion of the surfaces of particles that may be placed in contact with the fluid surface-treatment material. In some cases, the fluid surface-treatment material acts as a reservoir of a large number of molecules or other species such that attachment of some of those molecules or other species from the reservoir onto the surfaces of the particles does not significantly lower the concentration of those molecules or other species in the fluid-surface treatment material. This can be desirable since the same fluid surface-treatment material can be re-used repeatedly to alter the surfaces of particles in several different steps or re-used to modify the surfaces of other particles.

In some embodiments of the current invention, more than one species of molecule or other colloidal structure can be attached to the same patch or portion of the surface of the particles. This diversification of the species attached to a patch or portion of the surface of the particles can be obtained through just one surface-treatment step, or it can be obtained through two or more successive surface treatment steps. In some cases, it is desirable to have more than one type of molecule or species attached to the same localized area on the surfaces of the particles.

### Sample Embodiments

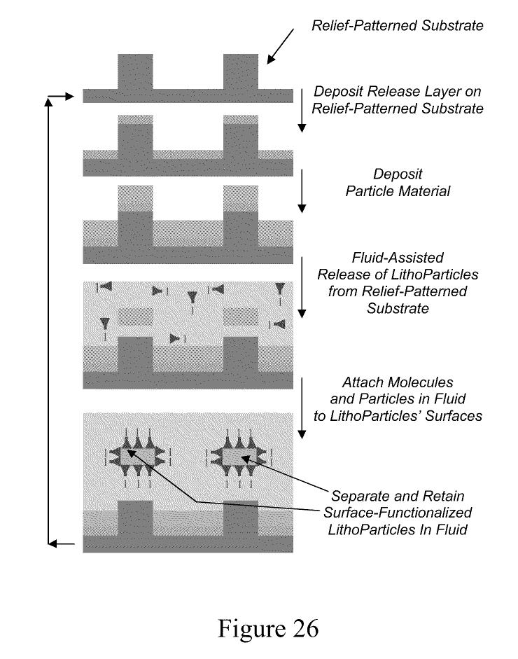

Sample embodiments of the present invention are shown in FIGS. 26-49 and provide a set of schematics for detailed procedures for how to obtain simple and complex surface modifications of particles made using relief deposition templating. It can be readily anticipated that the processes that are shown can be extended to create even more complicated surface modifications that would selectively provide for either shear-rigid or slippery bonding of a specific site on the surface of one particle with a specific site on the surface of a second particle.

In the example embodiments related to the steps for creating the surface-modified particles (FIGS. 26-45), the molecules or other species attached to the surfaces of the particles are labeled numerically by type of molecule or other colloidal species. Although “other species” is not always specifically mentioned along with “molecules” in these sample embodiments, the use of the word “molecules” often implies both molecules and other colloidal objects (e.g. nanoparticles). LithoParticle(™) refers to a shape-designed particle that is made using lithographic methods, including particles made by RDT.

In the four example embodiments related to the steps for assembling the surface-modified particles (FIGS. 46-49), the utility and design of the surface modification provides a means for controlling the structure of two-component and multi-component assemblies. In these examples, we show that it is possible to direct the assembly of particles to create desired relative positions and orientations of the particles, or desired ranges of relative positions and ranges of desired relative orientations. This approach enables a highly parallel method of mass-producing desired multi-component assemblies due to the controlled interactions between surface-modified particles in a fluid medium. Indeed, it can be readily anticipated that the assembly of surface-modified particles can be used to create a whole set of microscopic and nanoscopic mechanical linkages (Wikipedia article on mechanical linkages: http://en.wikipedia.org/wiki/Linkage_(mechanical); Schlater et al., Mechanisms and Mechanical Devices, 4th ed., McGraw-Hill, 2006). For instance, a smaller patch can slide on the surface of a larger patch to provide a desired range of motion(s) or degree(s) of freedom. Although our examples are for plate-like particles, it can be readily anticipated that the process of surface modification of particles and assembly of those particles can be used to assemble non-plate-like particles as well.

By incorporating surface treatment into the steps of the fabrication of shape-designed particles using RDT in a non-obvious and non-trivial manner, invention provides control for the attachment of molecules or other colloidal species to specific desired portions of the surfaces of the lithographic particles. This invention provides a well-defined method for causing the highly parallel assembly of many particles in solution into multi-component structures (e.g. small-scale devices or machines) through a set of well-defined steps by changing temperature, ionic strength, or other parameters that induces attractive interactions only between sites on the surfaces of the particles that have been specifically designed to bind together for certain ranges of conditions and not for others.

Post-Release Attachment of One Material onto LithoParticles' Surfaces

This exemplary embodiment is shown in FIG. 26. This example is shown for pillar template, but a well template could also be used. A single molecular or particle species is suspended or dispersed in the release fluid. These molecules or particles adsorb and attach to the exposed particle surfaces. This approach can create a surface coating uniformly distributed over the surfaces of the LithoParticles. The substrate need not be present after particle release for the surface modification of the particles to occur.

Post-Release Attachment of More than One Material onto LithoParticles' Surfaces

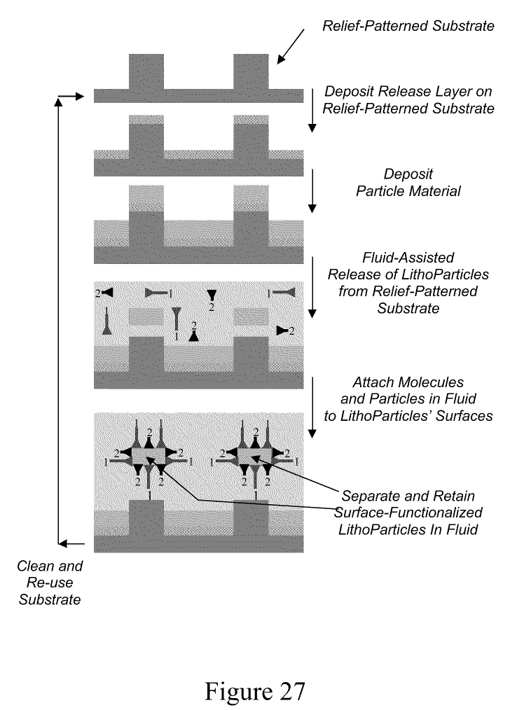

This exemplary embodiment is shown in FIG. 27. This example is shown for pillar template, but a well template could also be used. Molecular or particle species are suspended or dispersed in the release fluid. These adsorb and attach to the exposed particle surfaces. This approach can create a surface coating in which two or more species are uniformly distributed over the surfaces of the LithoParticles. The substrate need not be present after particle release for the surface modification of the particles to occur.

Post-Release Attachment of One or More Materials onto LithoParticles' Surfaces: Permanent Release Layer

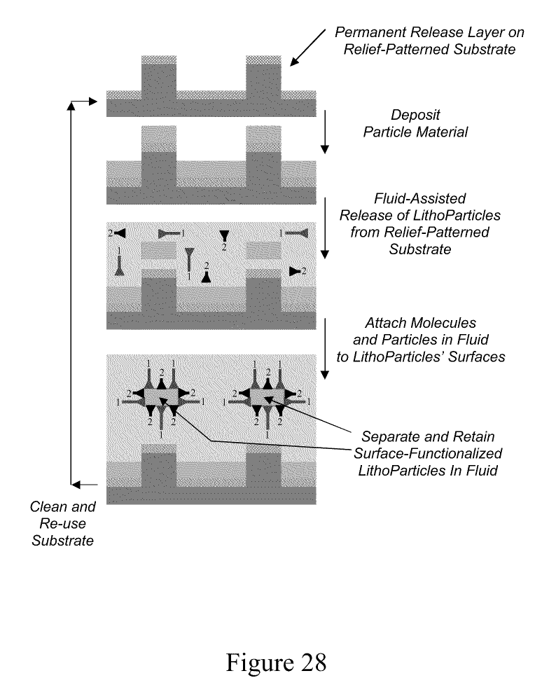

This exemplary embodiment is shown in FIG. 28. This example is shown for pillar template, but a well template could also be used. Molecular or particle species are suspended or dispersed in the release fluid. These adsorb and attach to the exposed particle surfaces. This approach can create a surface coating in which two or more species are uniformly distributed over the surfaces of the LithoParticles. The substrate need not be present after particle release for the surface modification of the particles to occur.

Post-Release Attachment of One or More Materials onto LithoParticles' Surfaces

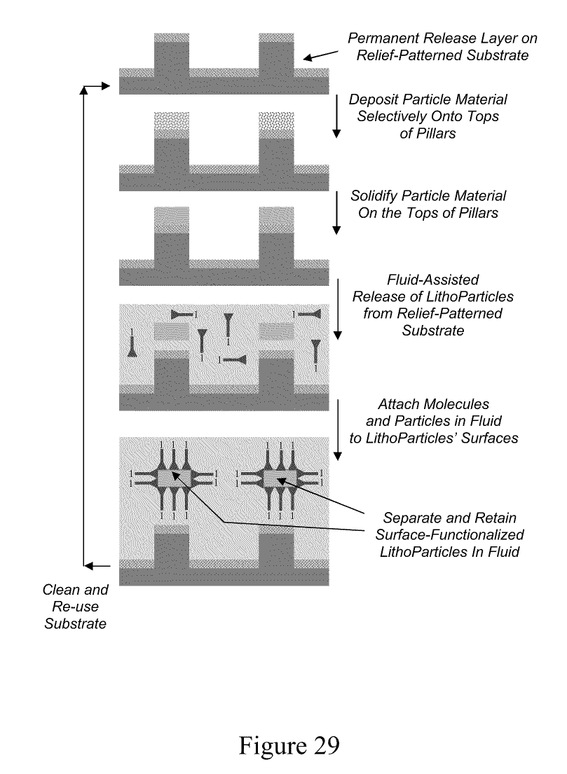

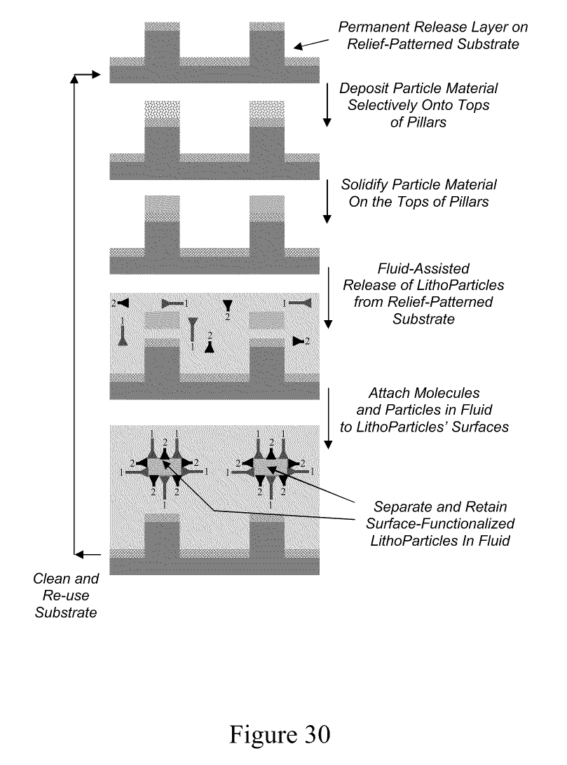

This exemplary embodiment is shown in FIGS. 29 and 30. This example takes advantage of the wetting of only the tops of the pillars that is common when a liquid material is coated onto a pillar template. The advantages of this method are: the particle material is deposited only in the regions that will lead to the desired particles, so the particle material is more efficiently used, and cleaning the substrate is easier at a later stage in the process. Molecular or particle species are suspended or dispersed in the release fluid. These adsorb and attach to the exposed particle surfaces. This approach can create a surface coating in which one or more species are uniformly distributed over the surfaces of the LithoParticles.

Pre-Release Attachment of One Material onto Portion of LithoParticles' Surfaces

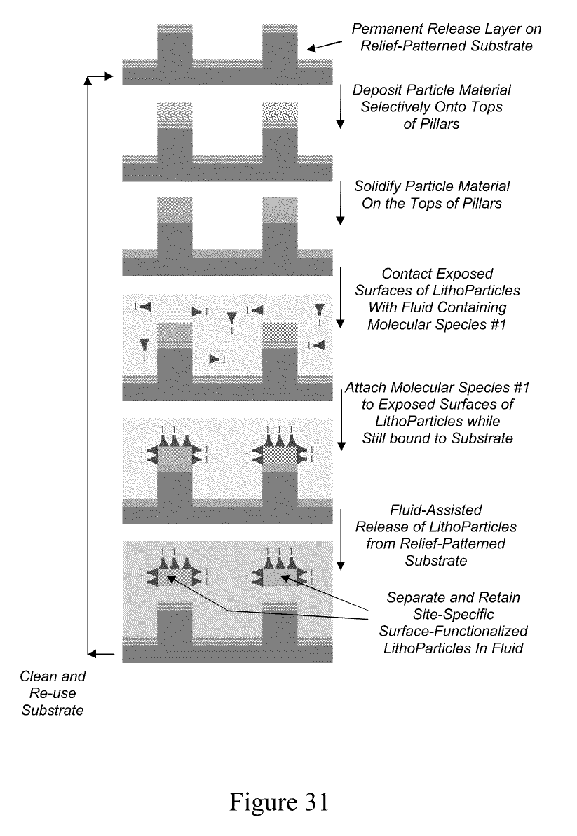

This exemplary embodiment is shown in FIG. 31. This example takes advantage of the wetting of only the tops of the pillars that is common when a liquid material is coated onto a pillar template. The advantages of this method are: the particle material is deposited only in the regions that will lead to the desired particles, so the particle material is more efficiently used, and cleaning the substrate is easier at a later stage in the process. Molecular or particle species are suspended or dispersed in the release fluid. These adsorb and attach to the exposed particle surfaces. This approach can create a surface coating in which one or more species are uniformly distributed over the surfaces of the LithoParticles.

Pre-Release and Post-Release Attachment of Two Different Materials onto Specific Portions of LithoParticles' Surfaces

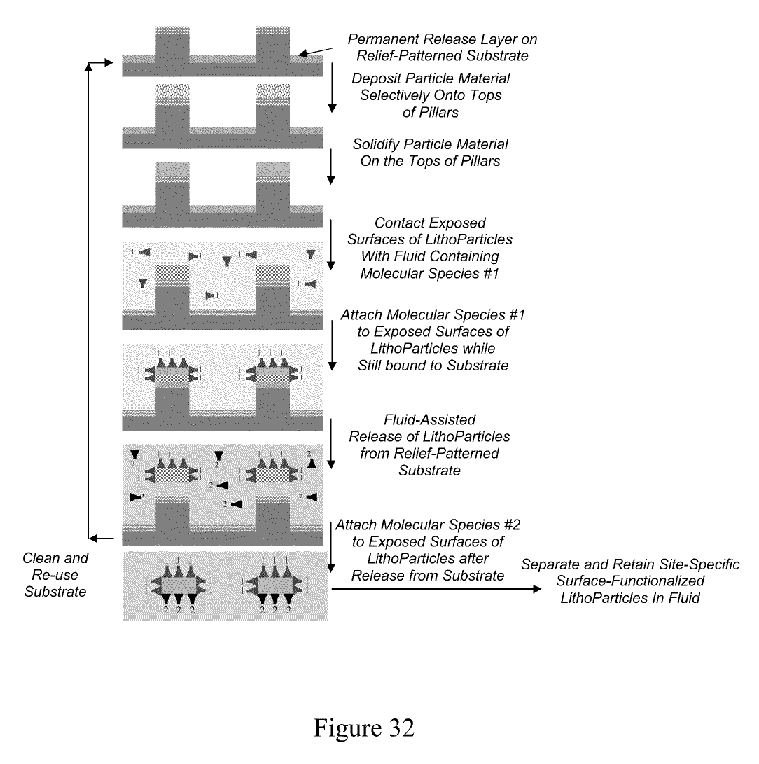

This exemplary embodiment is shown in FIG. 32. This example takes advantage of the wetting of only the tops of the pillars that is common when a liquid material is coated onto a pillar template. The advantages of this method are: the particle material is deposited only in the regions that will lead to the desired particles, so the particle material is more efficiently used, and cleaning the substrate is easier at a later stage in the process. Molecular or particle species are suspended or dispersed in the release fluid. These adsorb and attach to the exposed particle surfaces. This approach can create a surface coating in which two different materials are adsorbed to specific portions of the LithoParticles' Surfaces.

Post-Release Attachment of Two Different Materials onto Site-Specific Portions of Bilayer LithoParticles' Surfaces

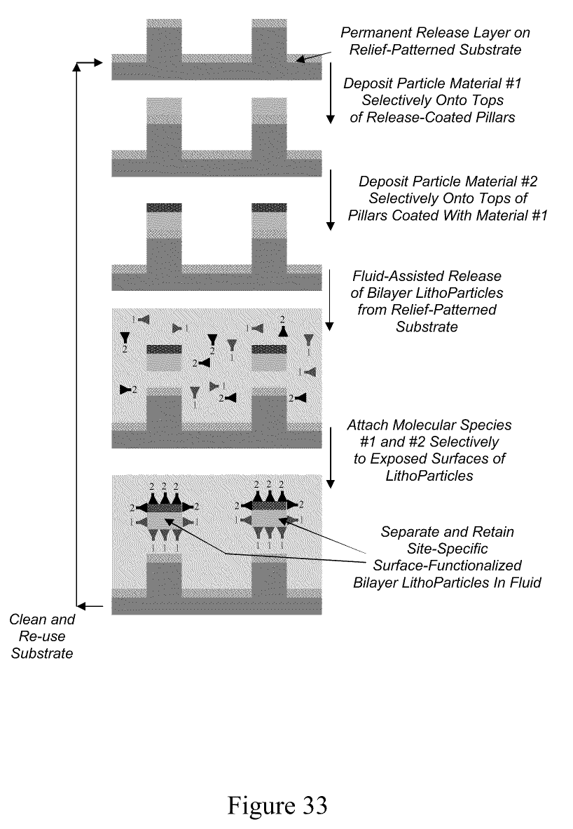

This exemplary embodiment is shown in FIG. 33. This example takes advantage of the wetting of only the tops of the pillars that is common when a liquid material is coated onto a pillar template. Use of two different types of molecules or particle attachment chemistries that depend on the type of material enable two different types of molecules or particles to diffuse, adsorb, and attach to specific portions of the surfaces of LithoParticles either before or after release. The case shown here is for post-release surface modification. Molecule type #1 attaches to the first Particle Material. Molecule type #2 attaches to the second Particle Material. These molecules can be in the release fluid or a separate coating step can be performed.

Pre-Release and Post-Release Attachment of Three Different Materials onto Site-Specific Portions of Bilayer LithoParticles' Surfaces

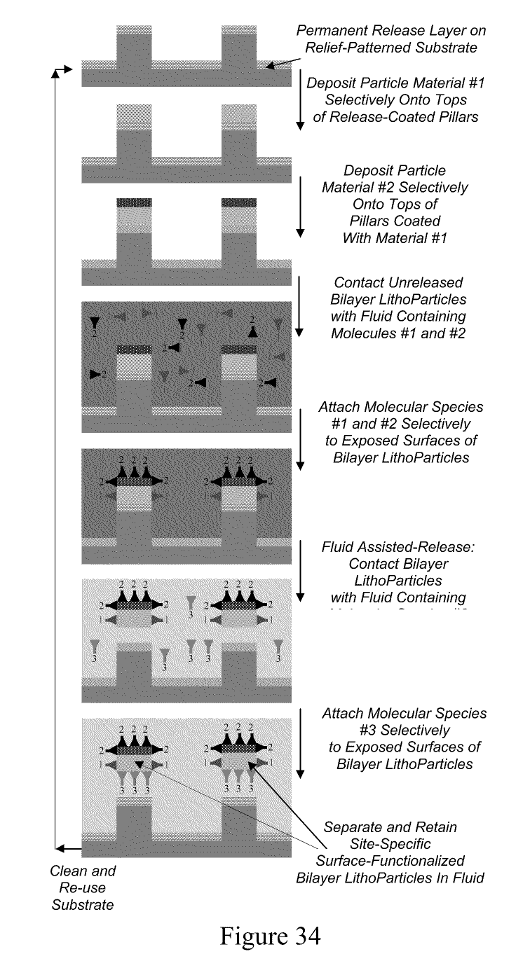

This exemplary embodiment is shown in FIG. 34. This method relies upon a combination of (1) using different particle materials that either bind or do not bind certain molecular types, depending upon the specific interactions of the molecules with the surfaces and (2) controlling which surfaces of the particles are exposed to the molecules before and after lift-off. Molecule types #1 and #3 attach only to the first Particle Material. Molecule type #2 attaches only to the second Particle Material (purple). These molecules can be in the release fluid or a separate coating step can be performed.

### Complex Hybrid Bilayer LithoParticles Made Using Two Relief Levels on the Top Pillar Surfaces

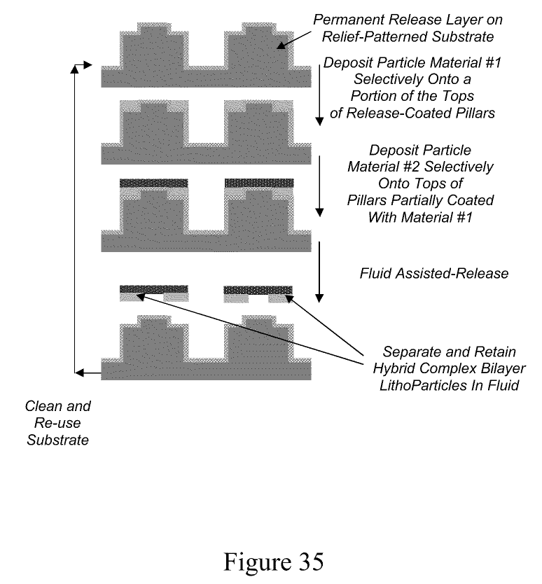

This exemplary embodiment is shown in FIG. 35. This approach provides a means for making LithoParticles that have different material compositions in designed locations. The relief of the pillars and the deposition of two or more different particle materials provides the spatial separation of the different material regions within a given LithoParticle. This spatial control can be used later in combination with molecule or particulate attachment chemistries that are specific to the material composition to which they can bind. In the example shown, cup-shaped LithoParticles are formed. The bottom of the cup is comprised of particle material #2 and the sides of the cup are comprised of particle material #1.

Pre-Release Attachment of Two Different Materials onto Site-Specific Portions of Bilayer LithoParticles' Surfaces

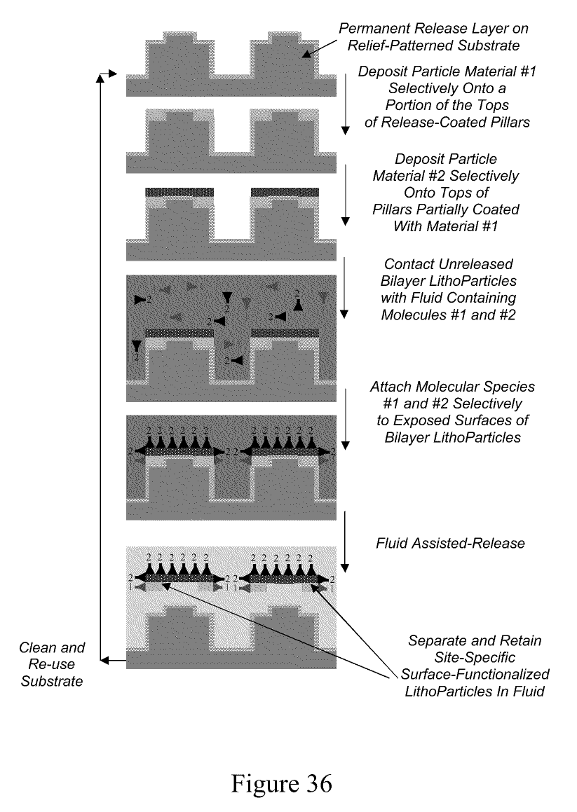

This exemplary embodiment is shown in FIG. 36. This approach creates exposed surfaces of two different particle material types so that different binding chemistries can be employed to attach molecules or particles selectively to specific regions of the surfaces of the LithoParticles. In this case, two different molecules are bound to the exposed surfaces of the particles prior to lift-off. This method relies upon a combination of (1) using different particle materials that either bind or do not bind certain molecular types, depending upon the specific interactions of the molecules with the surfaces and (2) controlling which surfaces of the particles are exposed to the molecules before and after lift-off. Molecule type #1 attaches to the first Particle Material (orange). Molecule type #2 attaches to the second Particle Material (purple). These molecules can be in the release fluid or a separate coating step can be performed.

Pre-Release and Post-Release Attachment of Four Different Materials onto Site-Specific Portions of Surfaces of LithoParticles Comprised of Two Particle Materials

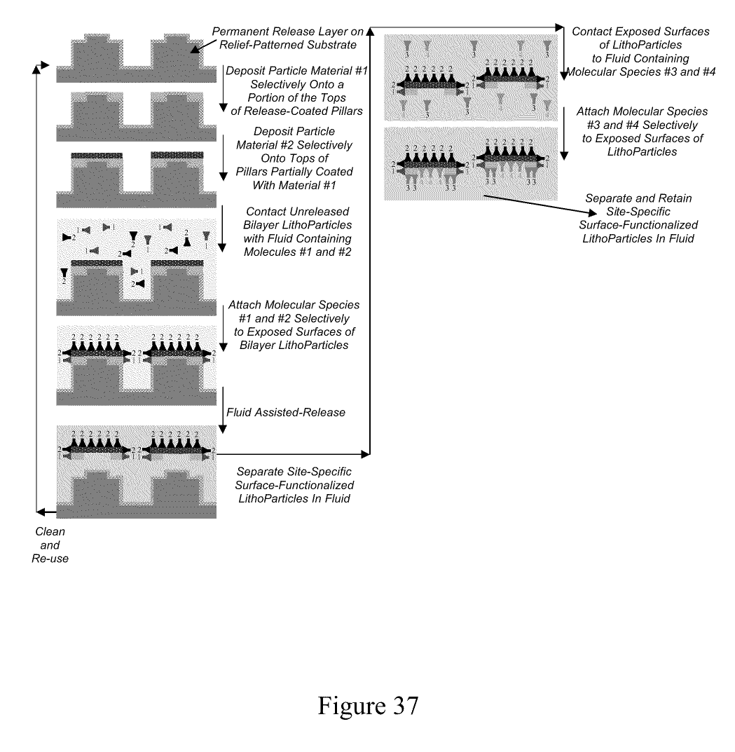

This exemplary embodiment is shown in FIG. 37. This method relies upon a combination of (1) using different particle materials that either bind or do not bind certain molecular types, depending upon the specific interactions of the molecules with the surfaces and (2) controlling which surfaces of the particles are exposed to the molecules before and after lift-off. Molecule types #1 and #3 attach only to the first Particle Material (orange). Molecule types #2 and #4 attach only to the second Particle Material (purple). These molecules can be in the release fluid or a separate coating step can be performed.

### Variation: Site-Selective Modification of a Portion of the Surfaces of LithoParticles Made Using Well-Deposition Templating

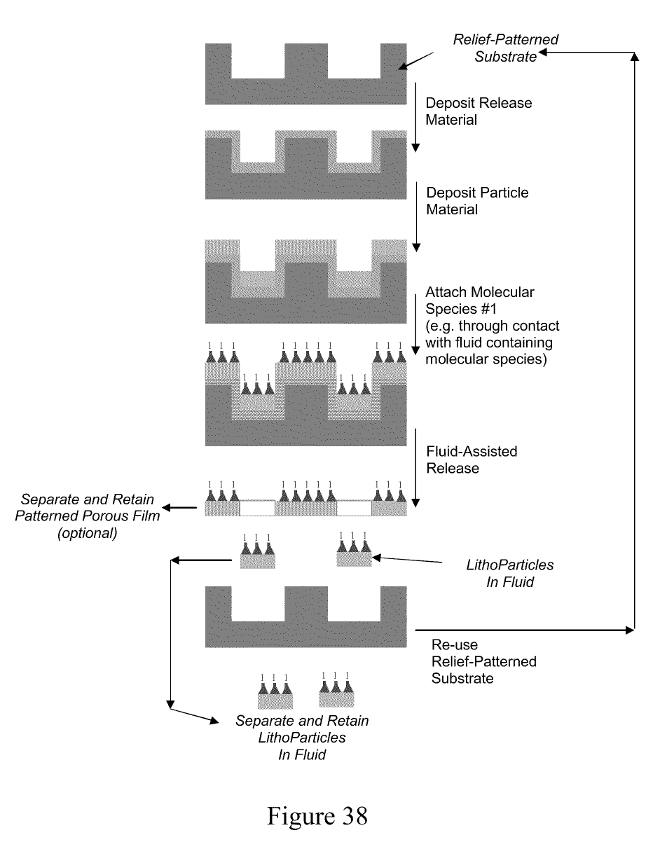

This exemplary embodiment is shown in FIG. 38. All of the methods described herein can also be implemented with negative-relief templates (i.e. well-templates). This example shows how molecules can be attached selectively to the exposed surfaces of particles in wells before the particles are released into a fluid, separated, and retained.

Incorporating an Internal Layer of Microscale or Nanoscale Particulates into LithoParticles

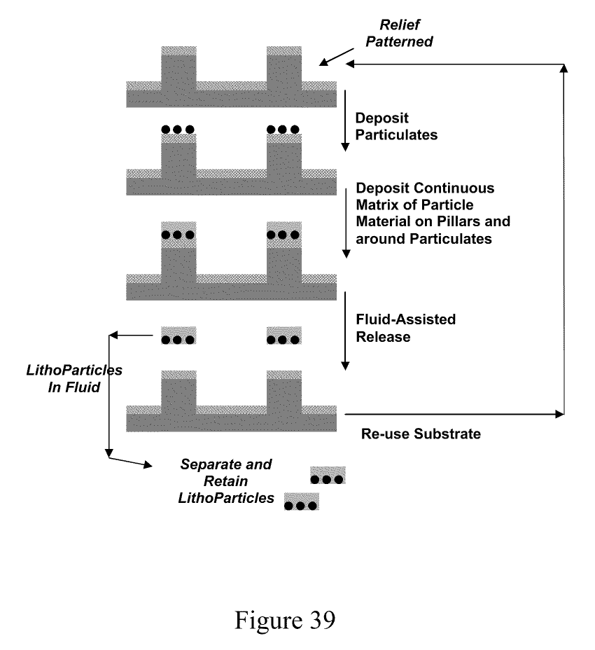

This exemplary embodiment is shown in FIG. 39. Deposition of particulates onto the surface of the release material can be uniform or non-uniform at low or high surface density. The particulates remain on the surface of the release layer as the continuous matrix of particle material is deposited to form the LithoParticles on the tops of the pillars. These LithoParticles contain particulates embedded inside in the form of a layer. Optionally, particulates can also be deposited on the surfaces of the lower trenches, but this will just result in more waste of the particulates since they will not be incorporated into the LithoParticles.

### Incorporating an External Layer of Microscale or Nanoscale Particulates on Exposed Surfaces of LithoParticles

These LithoParticles contain an inhomogeneous distribution of particulates on the outside in the form of a layer that imparts a surface roughness. The particulates stick and bind to the surface of the particle material layer after the deposition of the continuous matrix of particle material onto the tops of the pillars. Deposition of particulates onto the surface of the release material can be uniform or non-uniform at low or high surface density. By changing the type and size of the particulates, the surface roughness of the LithoParticles can be accurately controlled. Controlling the surface roughness on different portions of the surfaces of LithoParticles can be used to control site-selective assembly of the LithoParticles. Typically, the particulates are smaller than the size of the LithoParticles, but this is not a necessary condition. Optionally, particulates can also be deposited on the surfaces of the lower trenches, but this will just result in more waste of the particulates since they will not be incorporated into the LithoParticles.

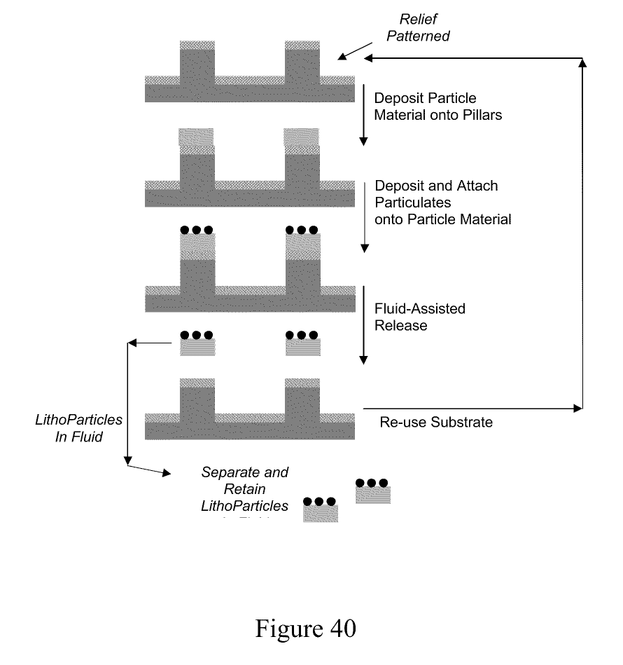

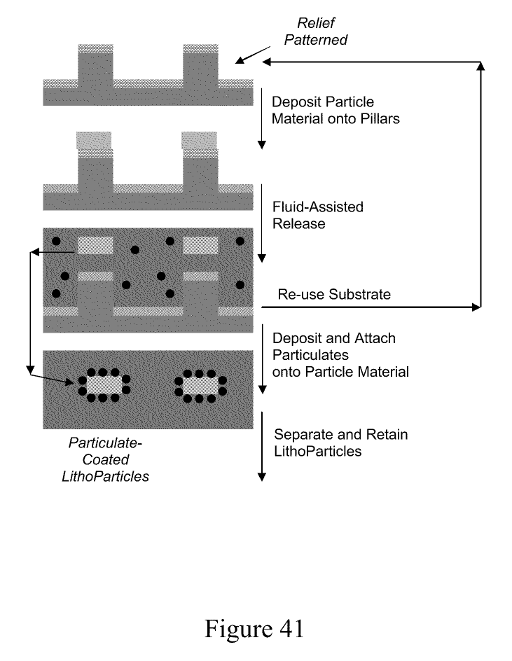

Incorporating an external layer may be performed before release of the LithoParticles, as shown in FIG. 40 to deposit particulates in specific regions. The incorporation may also be performed after release, as shown in FIG. 41 to deposit particulates over the whole surface.

### Pre-Release and Post-Release Shape Modification of LithoParticles: Rounding Sharp Edges and Corners

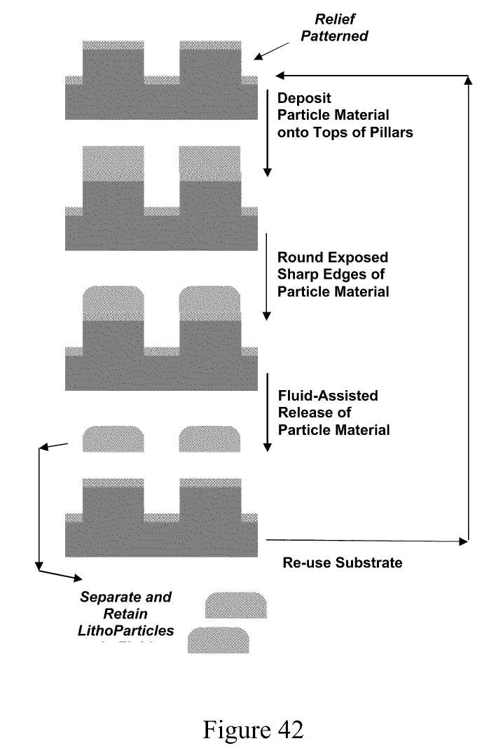

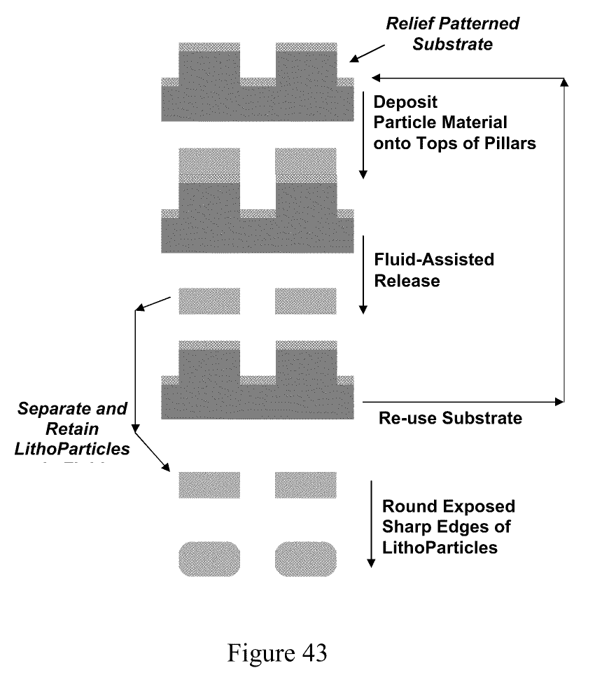

This exemplary embodiment is shown in FIGS. 42 and 43. The particle material is exposed to a chemical solution, external field, or is heated in such a way as to round the exposed sharp edges. This may occur before (FIG. 41) or after (FIG. 42) release of the LithoParticles. Note here that the word expose is used in two different senses in this procedural description: (1) exposing particle material to patterned radiation (2) exposed surfaces of particles that can be rounded.

Size and Shape Modification of LithoParticles: Coating LithoParticles in Fluid with a Second Type of Particle Material

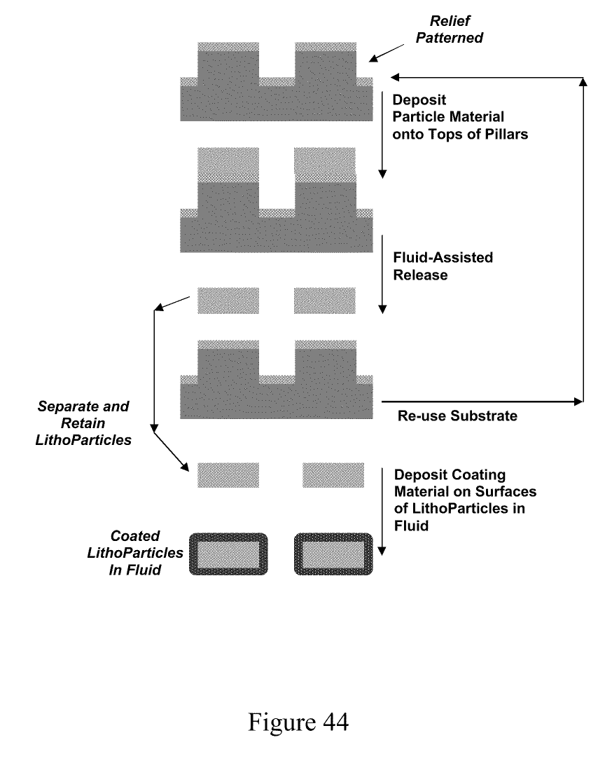

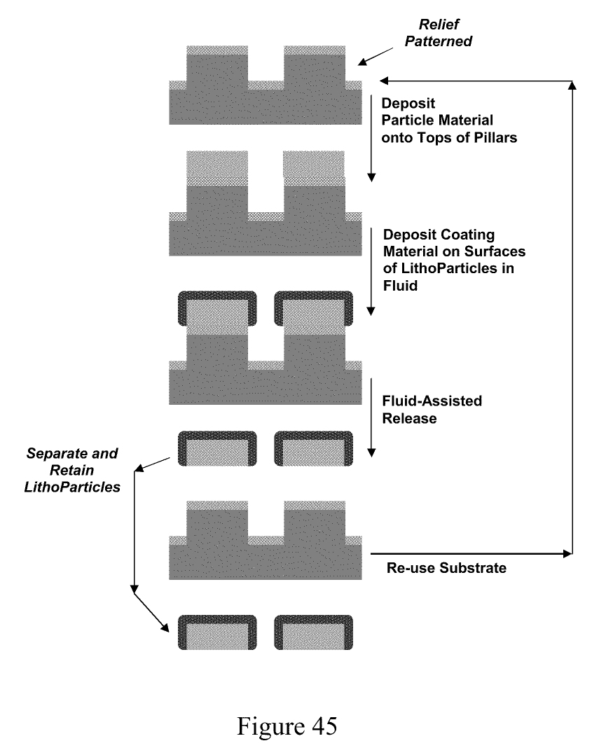

The LithoParticles are coated with a different particle material in a manner that can change the size and shape of the LithoParticles. Coating material can consist, for example, of monomers or oligomers that can be polymerized onto the surfaces of the released LithoParticles using known methods for making polymer spheres. This coating material can be directly applied through mechanical contact of a liquid to the surfaces of the particle material. The coating material can also be contained in a fluid solvent and essentially grown on the exposed surfaces of the particle material. Thin coatings may significantly alter the surface properties of the LithoParticles without altering their sizes and shapes much. Thick coatings may alter the surface properties of the LithoParticles and also their sizes and shapes quite significantly. Size and shape modification may occur after release of the LithoParticles (FIG. 44) or before release of the LithoParticles (FIG. 45)

### Directed Assembly of Surface-Modified Particles: Example 1

This exemplary embodiment is shown in FIG. 46. A square green patch (G) is a portion of the surface of a rectangular plate-like particle that has been modified by attaching species 1. A square blue patch (B) is a portion of the surface of a rectangular plate-like particle that has been modified by attaching species 2. In this example, a strong slippery attraction forms between a green patch and a blue patch when they approach into close proximity (i.e. energy of attraction is significantly stronger than thermal energy. All other surface interactions are repulsive or “hard” (e.g. green-green, blue-blue, grey-grey, grey-blue, grey-green). Particles are dispersed in a fluid material (not shown) and change configurations due to entropic fluctuations and/or applied external forces and torques. When a green patch comes in proximity with a blue patch, the attraction between the two patches causes the two particles to bond into a two-component structure (i.e. a dimer). Due to the slippery nature of the bond, thermal or applied torques cause the rectangles to rotate as they remain connected until a second green patch encounters a second blue patch, as shown. Consequently, relative rotations of the two particles cease, and a desired permanent angle of relative orientation of two identical particles is achieved. This example shows in detail how a two-component assembly of particles can be prescribed in a very precise manner by controlling the location and types of interactions between patches on particles in the fluid medium. This basic process can be extended to form multicomponent structures, not only simple dimers.

### Directed Assembly of Surface-Modified Particles: Example 2

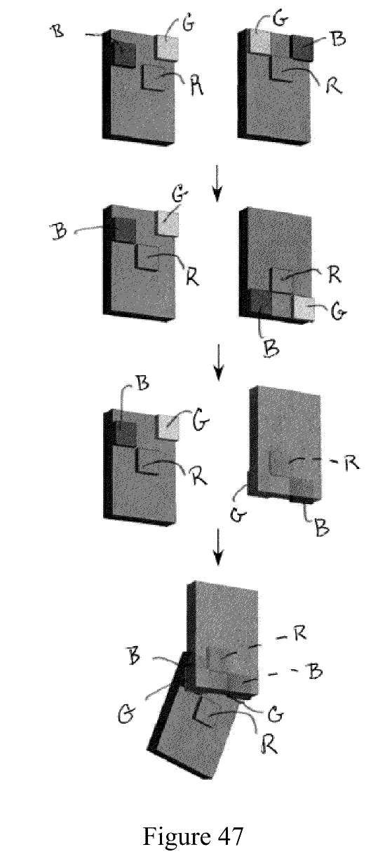

This exemplary embodiment is shown in FIG. 47. A square green patch (G) is a portion of the surface of a rectangular plate-like particle that has been modified by attaching species 1. A square blue patch (B) is a portion of the surface of a rectangular plate-like particle that has been modified by attaching species 2. A square red patch (R) is a portion of the surface of a rectangular plate-like particle that has been modified by attaching species 3. In this example, a strong slippery attraction forms between a green patch and a blue patch when they approach into close proximity (i.e. energy of attraction is significantly stronger than thermal energy). All other surface interactions are repulsive (red-red) or “hard” (e.g. green-green, blue-blue, grey-grey, grey-blue, grey-green, red-grey, red-blue, red-green). Particles are dispersed in a fluid material (not shown) and change configurations due to entropic fluctuations and/or applied external forces and torques. When a green patch comes in proximity with a blue patch, the attraction between the two patches causes the two particles to bond into a two-component structure (i.e. a dimer). Due to the slippery nature of the bond, thermal or applied torques cause the rectangles to rotate as they remain connected until a second green patch encounters a second blue patch, as shown. (Consequently, relative rotations of the two particles cease, and a desired permanent angle of relative orientation is achieved. By positioning the patches at different places on the surfaces of the rectangular particles, different permanent angles of relative orientation between the two particles can be directed through this assembly process. This example shows in detail how a two component assembly of particles can be directed in a very precise manner by controlling the location and types of interactions between patches on particles in the fluid medium.

### Directed Assembly of Surface-Modified Particles: Example 3

This exemplary embodiment is shown in FIG. 48. Circular green patch (G) is a portion of the surface of a rectangular plate-like particle that has been modified by attaching species 1. Circular blue patch (B) is a portion of the surface of a rectangular plate-like particle that has been modified by attaching species 2. In this example, we consider a strong slippery attraction between the green patch and the blue patch (i.e. energy of attraction is significantly stronger than thermal energy). All other surface interactions are repulsive or hard (e.g. green-green, blue-blue, grey-grey, grey-blue, grey-green). Particles are dispersed in a fluid material (not shown) and change configurations due to entropic fluctuations and/or applied external forces and torques. When a green patch comes in proximity with a blue patch, the attraction between the two patches causes the two particles to bond into a two-component structure (i.e. a dimer). Due to the slippery nature of the bond, torques around the axis formed by the centers of the circles do not experience elastic resistance, so the two rectangular particles can rotate into different relative angular orientations. This effectively forms a swivel joint that has a position determined by the location of the patches on the surfaces of the particles.

### Directed Assembly of Surface-Modified Particles: Example 4

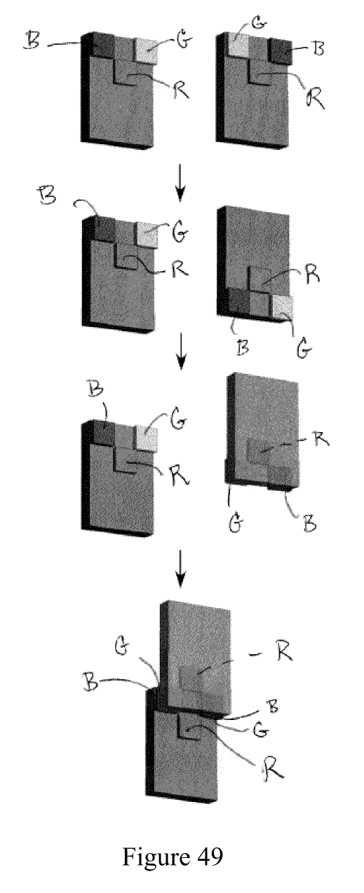

This exemplary embodiment is shown in FIG. 49. A square green patch (G) is a portion of the surface of a rectangular plate-like particle that has been modified by attaching species 1. A square blue patch (B) is a portion of the surface of a rectangular plate-like particle that has been modified by attaching species 2. A square red patch (R) is a portion of the surface of a rectangular plate-like particle that has been modified by attaching species 3. In this example, a strong slippery attraction forms between a green patch and a blue patch when they approach into close proximity (i.e. energy of attraction is significantly stronger than thermal energy). All other surface interactions are repulsive (red-red) or “hard” (e.g. green-green, blue-blue, grey-grey, grey-blue, grey-green, red-grey, red-blue, red-green). Particles are dispersed in a fluid material (not shown) and change configurations due to entropic fluctuations and/or applied external forces and torques. When a green patch comes in proximity with a blue patch, the attraction between the two patches causes the two particles to bond into a two-component structure (i.e. a dimer). Due to the slippery nature of the bond, thermal or applied torques cause the rectangles to rotate as they remain connected until a second green patch encounters a second blue patch, as shown. Consequently, relative rotations of the two particles cease, and a desired permanent angle of relative orientation of two identical particles is achieved. The repulsion of the red patches prevents a dimmer of a simple face-to-face orientation from forming. This example shows in detail how a two-component assembly of particles can be prescribed in a very precise manner by controlling the location and types of interactions between patches on particles in the fluid medium. This basic process can be extended to form multicomponent structures, not only dimers.

The current invention is not limited to the specific embodiments of the invention illustrated herein by way of example, but is defined by the claims. One of ordinary skill in the art would recognize that various modifications and alternatives to the examples discussed herein are possible without departing from the scope and general concepts of this invention.

We claim:

1. A method of producing at least one of microscopic and submicroscopic particles, comprising:
providing a template comprising a plurality of discrete surface portions, each discrete surface portion having a surface geometry selected to impart a desired geometrical property to a particle while being produced;
depositing a constituent material of said at least one of microscopic and submicroscopic particles being produced onto said plurality of discrete surface portions of said template to form at least portions of said particles;
separating said at least one of microscopic and submicroscopic particles comprising said constituent material from said template into a fluid material, said particles being separate from each other at respective discrete surface portions of said template; and
processing said template for subsequent use in producing additional at least one of microscopic and submicroscopic particles,
wherein said method of producing at least one of microscopic and submicroscopic particles is free of bringing a solid structure, other than said constituent material, into contact with said template proximate said plurality of discrete surface portions during said producing, and
wherein said method of producing at least one of microscopic and submicroscopic particles is free of bringing said solid structure into contact with said constituent material during said producing.

2. A method of producing particles according to claim 1, wherein said depositing is a directional deposition that leaves at least a fraction of said wall portion uncoated by said constituent material.

3. A method of producing particles according to claim 1, wherein said depositing is at least one of spin-coating, spray-coating, dip-coating, sputtering, vapor condensation, chemical vapor deposition, physical vapor deposition, laser ablation deposition, molecular beam epitaxy, electro-coating, and electron-beam metal evaporation.

4. A method of producing particles according to claim 1, wherein said depositing a constituent material of said at least one of microscopic and submicroscopic particles comprises at least one of depositing a material comprising at least one of a dispersion in a liquid of at least one of non-volatile molecules, polymeric materials, emulsions, nanoemulsions, surfactants, detergents, wetting agents, particles, atomic clusters, molecular clusters, organic particles, inorganic particles, metallic particles, nanoparticles, organic nanoparticles, inorganic nanoparticles, metallic nanoparticles, quantum dots, metal clusters, ferromagnetic particles, ferromagnetic nanoparticles, paramagnetic particles, paramagnetic nanoparticles, reactive molecules, radioactive isotopes, molecules containing radioactive isotopes, particles containing radioactive isotopes, nanoparticles containing radioactive isotopes, radiation-reactive molecules, derivatized molecules, fluorescent molecules, dye molecules, drug molecules, biomolecules biologically active molecules, proteins, lipids, deoxyribonucleic acids, ribonucleic acids, single-stranded deoxyribonucleic acid oligomers, partially single-stranded deoxyribonucleic acid oligomers peptides, polypeptides and any combination thereof; and at least one of solidifying, reacting, linking, bonding, aggregating, gelling, entangling, sintering, evaporating, freezing, or baking at least a portion of said constituent material subsequent to said depositing.

5. A method of producing particles according to claim 1, wherein said providing a template provides a template comprising a plurality of wells, each well being a low-surface portion of said template defined by a surrounding high-surface portion of said template and a wall portion therebetween, said surrounding high-surface portion being a contiguous surface around respective peripheries of all of said plurality of wells.

6. A method of producing particles according to claim 5, wherein said depositing constituent material deposits constituent material that substantially fills said plurality of wells and deposits a layer of constituent material on said high-surface portion surrounding said plurality of wells.

7. A method of producing particles according to claim 6, further comprising:
removing said layer of constituent material from said high-surface portion surrounding said plurality of wells; and
separating a plurality of particles from said template.

8. A method of producing particles according to claim 1, wherein said providing a template provides a template comprising a plurality of pillars, each pillar being a high-surface portion of said template defined by a surrounding low-surface portion of said template and a wall portion therebetween, said surrounding low-surface portion being a contiguous surface around respective peripheries of all of said plurality of pillars.

9. A method of producing particles according to claim 5, wherein said providing a template provides a template comprising a plurality of pillars, each pillar being a high-surface portion of said template defined by a surrounding low-surface portion of said template and a wall portion therebetween, said surrounding low-surface portion being a contiguous surface around respective peripheries of all of said plurality of pillars.

10. A method of producing particles according to claim 1, wherein said providing a template provides a template comprising a coating of a material that facilitates said separating said at least one of microscopic and submicroscopic particles.

11. A method of producing particles according to claim 10, wherein said separating said at least one particle comprises removing said coating of material that facilitates said separating.

12. A method of producing particles according to claim 11, wherein said removing said coating comprises immersing said template in a fluid that acts to dissolve said coating.

13. A method of producing particles according to claim 11, wherein said removing said coating comprises heating said template to melt said coating.

14. A method of producing particles according to claim 10, wherein said separating said at least one particle comprises immersing said template in a fluid and agitating at least one of said template and said fluid to cause said separating said at least one particle while leaving said coating of material that facilitates said separating substantially unchanged.

15. A method of producing particles according to claim 8, wherein said depositing comprises dipping said pillars into said constituent material.

16. A method of producing particles according to claim 8, wherein said depositing comprises applying a voltage between said template and said constituent material.

17. A method of producing particles according to claim 1, wherein said depositing a constituent material of said particles being produced comprises depositing a plurality of layers of material, each layer having a different composition.

18. A method of producing particles according to claim 1, wherein said separating said particles provides particles having a maximum dimension less than about 1 mm.

19. A method of producing particles according to claim 1, wherein said separating said particles provides particles having a maximum dimension less than about 0.1 mm and greater than about 1 nm.

20. A method of producing particles according to claim 1, wherein said separating said particles comprises separating at least one hundred thousand particles prior to said processing said template for subsequent use in producing additional particles.

21. A method of producing particles according to claim 1, wherein said fluid material comprises a liquid material within which said particles produced form a dispersion after said separation.

22. A method of producing particles according to claim 21, further comprising adding to said liquid material in which said particles are dispersed at least one of an additive selected from the group of additives consisting of an acidic material, a basic material, an electrolyte material, an ionic material, a polar material, a non-polar material, a buffer, a surfactant, a lipid, a resin, a polymer, a block copolymer, a star polymer, a dendrimer, a wax, an oil, a juice, an extract, a flavor, a perfume, an aqueous solution, a biomolecule, a biopolymer, a microparticle, a nanoparticle, a droplet, a bubble, a foam, a dye, an ink, a paint, a fluorescent molecule, a pigment, a viscosity modifier, a stabilizer, a refractive index modifier, a thermal modifier, a surface energy modifier, a wetting modifier, a plasticizer, a swelling agent, a shrinking agent, a sol, a gel, a glass, an ion exchange resin, a nanoemulsion, a microemulsion, a thermotropic liquid crystal, a lyotropic liquid crystal, a clay, a bonding agent, an adhesion promoter, a liposome, a polymersome, a colloidosome, a vesicle, a micelle, a graphene material, a fullerene material, a nanotube, a nanosheet, a nanowire, a nucleic acid, a ribonucleic acid, a single-stranded deoxyribonucleic acid, a double-stranded deoxyribonucleic acid, an amino acid, a protein, a peptide, a polypeptide, an albumin, a collagen, a cellulose, a serum, an enzyme, an antibody, an antigen, an algenate, a biological cell, a biological tissue, a co-polypeptide, a vitamin, a nutrient, a biomolecular motor, a biomolecular assembly, a virus, a vault, a saccharide, a polysaccharide, a catalyst, an oligomeric molecule, a crosslinker molecule, an initator, and a quantum dot.

23. A method of producing particles according to claim 1, further comprising depositing a sacrificial coating of non-constituent material on said template prior to said depositing said constituent material thereon,
wherein said separating said at least one of microscopic and submicroscopic particles comprising said constituent material from said template into a fluid material comprises at least one of dissolving, sublimating, melting, eroding, and evaporating said sacrificial layer.

24. A method of producing particles according to claim 1, further comprising thermally processing said constituent material prior to said separating.

25. A method of producing particles according to claim 1, wherein said deposited constituent material has a maximum predetermined spatial dimension of thickness between about one nanometer and about ten micrometers.

26. A method of producing particles according to claim 1, wherein a maximum predetermined spatial dimension of each of said particles produced is less than about ten micrometers and more than about one nanometer.

27. A method of producing particles according to claim 1, wherein said separating includes liberating at least 1,000 particles from said template.

28. A method of producing particles according to claim 1, further comprising a deposition of at least one of a metallic material, an organic material, a magnetic material, a particulate material, and a composite material prior to said separating said particles.

29. A method of producing particles according to claim 1, wherein said template comprises at least one of a low surface-energy surface and a low surface-energy surface coating to facilitate said separating at least one particle.

30. A method of producing particles according to claim 1, wherein said separating comprises at least one of a mechanical agitation, a vibration, an acoustic agitation, an ultrasonic agitation, a temperature change, and a fluid flow to cause said particles to separate from said template.

31. A method of producing particles according to claim 1, wherein said particles comprise a material in a composition thereof that modifies at least one of an optical property, a magnetic property, an electrical property, a mechanical property, a radioactive property, a nuclear isotopic property, a biocompatibility property, a biodegradability property, a porosity property, a thermal property, a wetting property, a surface roughness property, a solubility property, and a catalytic property of said particles.

32. A method of producing particles according to claim 1, further comprising modifying a surface of said particles with a surface-modifying material having a predetermined chemical property by at least one of functionalizing, adsorbing, and coating said particles with said surface-modifying material after said separating.

33. A method of producing particles according the previous claim 32, wherein said modifying a surface of said particles with a surface-modifying material having a predetermined chemical property comprises stabilizing said particles to inhibit at least one of aggregation, agglomeration, and clumping.

34. A method of producing particles according to claim 32, wherein said surface-modifying material comprises a material selected from the group of materials consisting of a surfactant, an ionic surfactant, a cationic surfactant, a zwitterionic surfactant, a non-ionic surfactant, a polymeric surfactant, a lipopolymer, a lipid, a lipid bilayer, a lamellar vesicle, a multi-lamellar vesicle, a polymer, a derivatized polymer, a homopolymer, a copolymer, a block copolymer, a random copolymer, a polymer brush, a polymer coil, a polymer tether, a star polymer, a dendrimer, a polyacid, a polybase, a polyelectrolyte, a semiflexible polymer, a flexible polymer, a polyethylene glycol, a polysaccharide, a polyhydroxystearic acid, a polyvinylalcohol, a polysiloxane, a charge group, a sulfate group, a sulfonate group, a carboxylate group, an amine group, an acidic group, a basic group, a biomolecule, a biopolymer, a derivatized biopolymer, an antibody, an antigen, a peptide, a polypeptide, a copolypeptide, an amino acid, a protein, a membrane protein, a transcription protein, a structural protein, a snare protein, an actin, a tubulin, an enzyme, a vitamin, a biological cell wall, an albumin, a collagen, a cellulose, a cholesterol, a biomolecular motor, a kinesin, a saccharide, a liposaccharide, a biotin, a streptavidin, a nucleic acid, a ribonucleic acid, a deoxyribonucleic acid, a derivatized deoxyribonucleic acid, an oligomeric nucleic acid, an oligomeric single-stranded deoxyribonucleic acid, an oligomeric double-stranded deoxyribonucleic acid, a biomolecular assembly, a biomotor, an acidic material, a basic material, a metallic material, an inorganic material, and organic material, a polar material, a non-polar material, a particulate material, a microparticle, a nanoparticle, a droplet, a microdroplet, a nanodroplet, a chemically reactive material, a thermally reactive material, a photoreactive material, a photoabsorbing material, a catalytic material, an isotopic material, a radioactive material, a thiolated molecule, an alkane, a silane, and a siloxane.

35. A multi-component composition, comprising:
a first material component in which particles can be dispersed; and
a plurality of particles dispersed in the first material component,
wherein said plurality of particles are produced by the method of claim 1, and wherein said plurality of particles is at least 1,000 particles produced in a parallel process.

36. A multi-component composition according to claim 35, wherein said first material component is one of a liquid, a dispersion, a solution, an ink, or a paint, said multi-component composition providing at least one of a security-labeled ink, a security labeled paint, a biomarker, a nanobiomaterial, or an identifier label.

37. A system for manufacturing at least one of microscopic and submicroscopic particles, comprising:
a template cleaning and preparation system;
a deposition system arranged proximate said template cleaning and preparation system to be able to receive a template from said template cleaning and preparation system upon which material will be deposited to produce said particles; and
a particle removal system arranged proximate said deposition system to be able to receive a template from said deposition system after material has been deposited on said template,
wherein said system for manufacturing particles is free of a structural component, other than said constituent material, for contacting with said template proximate a plurality of discrete surface portions of said template, and
wherein said system for manufacturing particles is free of a structural component, other than said constituent material, for contacting with said constituent material during said producing.
